(19) United States
(12) Patent Application Publication
(10) Pub. No.: US 2015/0315654 A9
(43) Pub. Date: Nov. 5, 2015

(54) Chondroitin Sulfate Sulfotransferases and Proteoglycans as Cancer Biomarkers: Use of Expression and Methalytion Status

(71) Applicant: The Board of Trustees of the University of Arkansas, Little Rock, AR (US)

(72) Inventors: Behjatolah Karbassi, Little Rock, AR (US); Thomas Kieber-Emmons, Little Rock, AR (US); Aiwei Yao-Borengasser, Little Rock, AR (US); Damir Herman, Los Gatos, CA (US); Fariba Jousheghany, Little Rock, AR (US); Craig Anthony Cooney, Little Rock, AR (US)

(73) Assignee: The Board of Trustees of the University of Arkansas, Little Rock, AR (US)

(21) Appl. No.: 13/573,653

(22) Filed: Oct. 1, 2012

Related U.S. Application Data
(63) Continuation-in-part of application No. 13/440,137, filed on Apr. 5, 2012.
Application No. 13/440,137 is a division of application No. 12/286,950, filed on Oct. 4, 2008, now Pat. No. 8,173,103.
Application No. 12/286,950 is a continuation-in-part of application No. 11/694,370, filed on Mar. 30, 2007, now abandoned.
(60) Provisional application No. 61/626,646, filed on Oct. 1, 2011.
(60) Provisional application No. 60/788,018, filed on Mar. 31, 2006.

(51) Int. Cl. C12Q 1/68

(52) U.S. Cl. CPC C12Q 1/6886

(57) ABSTRACT

A method of determining a prognosis of a cancer in a human comprising: determining expression level of CHST11 in a cancer tissue sample or determining methylation status of CHST11 gene in a cancer tissue sample. CHST11 is Carbohydrate (Chondroitin 4) Sulfotransferase 11.

### CROSS-REFERENCES TO RELATED APPLICATIONS

This application claims priority under 35 U.S.C. 119 from U.S. Provisional application No. 61/626,646, filed Oct. 1, 2011, the contents of which are incorporated herein by reference in their entirety.

### STATEMENT REGARDING FEDERALLY SPONSORED RESEARCH

This invention was made with government support under a Clinical and Translational Science Award 1UL1RR029884 from the National Institutes of Health National Center for Advancing Translational Sciences. The government has certain rights in this invention.

### BACKGROUND

Tumor-associated glycans play a significant role in promoting aggressive and metastatic behavior of malignant cells [1-5], participating in cell-cell and cell-extracellular matrix interactions that promote tumor cell adhesion and migration. Among glycans that play a critical role in stromal tumor cell interactions are glycosaminoglycans (GAGs) attached to proteoglycans (PGs). Altered production levels of PGs and structural changes in their GAGs are reported in many neoplastic tissues [6-10]. GAGs are polysaccharide chains covalently attached to protein cores that together comprise PGs [6, 11] and based on the prevalence of GAG chains, chondroitin sulfate (CS)/dermatan sulfate (DS) PGs (CS/DS-PGs), heparan sulfate PGs and keratan sulfate PGs have been described [12]. Increased production of CS/DS-GAGs is found in transformed fibroblasts and mammary carcinoma cells [8, 13, 14] and it has been shown that these polysaccharides contribute to fibrosarcoma cell proliferation, adhesion and migration [15].

Several studies have disclosed the critical involvement of P-selectin in the facilitation of blood borne metastases [16-18]. P-selectin/ligand interaction often requires sialylated and fucosylated carbohydrate such as sialyl Lewis X and sialyl Lewis A [19]; however, P-selectin also binds to heparan sulfate, certain sulfated glycolipids and CS/DS-GAGs [20-23]. In previous studies we found that CS/DS-GAGs are expressed on the cell surface of murine and human breast cancer cell lines with high metastatic capacity and that they play a major role in P-selectin binding and P-selectin-mediated adhesion of cancer cells to platelets and endothelial cells [24]. However, variation in the abundance and function of CS/DS relative to tumor cell phenotypic properties and P-selectin binding are not well defined. It is likely that P-selectin binding to tumor cells and the functional consequences of such binding are dependent on which sulfotransferases define the relevant CS/DS and which core proteins carry the CS polysaccharide.

CS/DS expression is controlled by many enzymes in a complex biosynthetic pathway and this leads to considerable variation in structure and function. The chondroitin backbone of CS/DS-GAGs consists of repetitive disaccharide units containing D-glucuronic acid (GlcA) and N-acetyl-D-galactosamine (GalNAc) residues, or varying proportions of L-iduronic acid (IdoA) in place of GlcA [25, 26]. Major structural variability of the CS/DS chains is due to the sulfation positions in repeating disaccharide units by the site-specific activities of sulfotransferases that produce the variants CS-A, CS-B (dermatan sulfate, DS), CS-C, CS-D and CS-E [26, 27]. CHST3, CHST7, CHST11, CHST12, CHST13, CHST14 and CHST15 are the enzymes that sulfate the GalNAc residues of chondroitin polymers.

The variation, abundance and function of CS/DS-GAGs are also affected by the expression of the PG core protein presenting them. Syndecan-1 (SDC-1), syndecan-4 (SDC-4), neuropilin-1 (NRP-1) and CSPG4 are membrane proteins capable of carrying CS chains [35-39]. Among these PGs, CSPG4 exclusively carries CS chains [40, 41].

We and others have previously shown that glycosaminoglycans on tumor cells are involved in binding to P-selectins, and that this interaction is involved in metastasis.

There is a need for cancer markers to better indicate cancer prognosis. This can help guide treatment plans as well as help patients plan their lives and make treatment decisions.

### SUMMARY

Here we have investigated the utility of structural genes for glycosaminoglycans and genes encoding synthetases involve in glycosaminoglycan synthesis for possible utility as prognostic markers in cancer treatment.

It is shown here that the Carbohydrate (Chondroitin 4) Sulfotransferase 11 (CHST11) gene is highly expressed in aggressive breast cancer cells but significantly less so in less aggressive breast cancer cell lines. Furthermore, a positive correlation was observed between the expression levels of CHST11 and in vitro indicators of metastasis.

The same observations were made concerning the carrier proteoglycan Chondroitin Sulfate Proteoglycan 4 (CSPG4): It is highly expressed on aggressive breast cancer cell lines, and less so on less aggressive breast cancer cell lines; and its overexpression contributes to in vitro indicators of metastasis.

In addition, CSPG4 and CHST11 were over-expressed in tumor-containing clinical tissue specimens compared with normal tissues.

Thus, overexpression of either or both of CHST11 and CSPG4 genes is predictive of more aggressive tumors and indicates a worse prognosis.

We have also found that expression level in tumor samples of both the CHST11 and CSPG4 genes is dependent on the methylation state of these genes. Specifically, both the CHST11 and CSPG4 genes are overexpressed in tumors when the CHST11 and CSPG4 genes are hypomethylated in the tumor tissue. Thus, hypomethyation of these genes can be used as a surrogate indicator of overexpression of the two genes. This means that overexpression of the CHST11 and CSPG4 genes can be inferred in a tumor when the tumor tissue has these genes hypomethylated. Direct measurement of gene expression requires living tumor cells or well prepared frozen samples, but paraffin-embedded sections and catalogued stained tissues, can be used for DNA samples that will show whether these genes are hypomethylated or hypermethylated. Thus, in some cases it is easier to determine methylation state of these genes than to directly measure overexpression, and many tissue samples can be used to determine methylation status that cannot be used for measurement of gene overexpression.

One embodiment of the invention provides a method of determining a prognosis of a cancer in a human comprising: determining expression level of CHST11 in a cancer tissue sample or determining methylation status of CHST11 gene in a cancer tissue sample. High expression indicates a more aggressive tumor and a tumor more likely to metastasize.

Another embodiment of the invention provides a method of determining a prognosis of a cancer in a human comprising: determining expression level of CSPG4 in a cancer tissue sample or determining methylation status of CSPG4 gene in a cancer tissue sample. High expression indicates a more aggressive tumor and a tumor more likely to metastasize.

Another embodiment provides a method of determining a prognosis of a cancer in a human comprising: (a) determining expression level of CHST11 in a cancer tissue sample or determining methylation status of CHST11 gene in a cancer tissue sample and (b) determining expression level of CSPG4 in a cancer tissue sample or determining methylation status of CSPG4 gene in a cancer tissue sample.

### BRIEF DESCRIPTION OF THE DRAWINGS

FIG. 1. The Expression of the Chondroitin Sulfotransferase Genes, in the Breast Cancer Cells Used.

The expression of CHST3, CHST7, CHST11, CHST12, CHST13, CHST14, CHST15 was measured by qRT-PCR and normalized using 18S. Data were analyzed by one-way ANOVA and post hoc analysis using data from three (CHST7, CHST12, CHST13, CHST14) and four (CHST3, CHST11, CHST15) independent experiments. Means and standard deviations are shown. Statistically significant differences and P values are shown.

FIG. 2. Anti-CS-A mAb and P-Selectin Showed Comparable Binding to the Cell Lines.

A) Flow cytometry analysis of CS-A expression using anti-CS-A 2H6 mAb (15 μg/ml). B) P-selectin binding to cells using recombinant P-selectin (15 μg/ml). Open histograms show binding of the secondary antibody only (control), while filled histograms show anti-CS-A and P-selectin binding. One representative experiment out of three is shown.

FIG. 3. Inhibition of CHST11 Expression and P-Selectin Binding to MDA-MB-231 Cells by CHST11 siRNA.

MDA-MB-231 cells were treated with three different siRNAs for the CHST11 gene. RNA was harvested after 48 hours and gene expression was assayed at the mRNA level (A). GAPDH was used as the house keeping gene to normalize mRNA-based expression data using the delta delta CT method. CHST11 mRNA levels are shown relative to mRNA level in cells treated with transfection agent only (vehicle). Data were log transformed and subjected to one way ANOVA with post-hocTukey's analysis. B) Binding of anti-CS-A (2H6 mAb) (top) and P-selectin (bottom) was tested at Day 6 post siRNA transfection. Binding of secondary antibodies only serves as control. Binding of anti-CS-A 2H6 mAb and recombinant human P-selectin to vehicle-treated and siRNA-treated MDA-MB-231 cells with the three siRNAs is shown. Mean fluorescent intensities of three independent experiments were log transformed and analyzed by ANOVA and post-hoc comparison. Treatment with CHST11 siRNA #31 significantly reduced mean fluorescent intensities for anti-CS-A 2H6 mAb (P≦0.015) and P-selectin (P≦0.001) binding, as compared with vehicle-treated cells.

FIG. 4. The Expression of CSPG4 in Aggressive Breast Cancer Cell Lines Contributes to P-Selectin Binding.

A) CSPG4 mRNA was measured by qRT-PCR and normalized to 18S values and log transformed. Means and standard deviations are shown. Comparisons were made by ANOVA and post-hoc analysis. B) Cell surface expression of CSPG4 was examined in the indicated breast cancer cell lines by flow cytometry using anti-CSPG4 225.28 mAb (10 μg/ml). Open histogram shows binding of the secondary antibody only, while filled histogram shows anti-CSPG4 225.28 mAb binding. One representative experiment out of three is shown. C) Expression of CSPG4 was inhibited by transient transfection of MDA-MB-231 cells with CSPG4 siRNA that led to a decrease in the binding of P-selectin (D) and anti-CS-A (E) to transfected cells In C, D and E the filled histograms show the binding of secondary antibodies only (control), the open histograms with solid lines show binding to vehicle-treated cells while the open histograms with dotted lines show binding to the siRNA-transfected cells.

FIG. 5. Expression of CSPG4 Leads to an Increase in CS-A Expression and P-Selectin Binding.

A) Secondary Ab binding (control) to CSPG-4-transfected M14 (M14-CSPG4). Anti-CSPG4 (mAb 225.28) binding to M14-mock-transfected is minimal (B), while the binding is high to CSPG4-transfected cells (C). D) Overlay histogram of 2H6 mAb (anti-CS-A) binding to the M14-mock-transfected (filled histogram) and M14-CSPG4 cell line (open histogram). E) P-selectin binds to M14-CSPG4 (open histogram, solid line) and not to M14-mock-transfected (filled histogram). Binding of P-selectin to M14-CSPG4 is reduced after treatment with chondroitinase ABC (dotted line shifted to the left). The experiment was repeated three times and one representative is shown.

FIG. 6. Expression of the CSPG4 and the CHST11 Genes was Detected in Breast Tissue Samples.

mRNA expression was quantified by absolute quantification and the ratio of mRNA to 18S mRNA was calculated. The fold change in tumor sample compared to normal tissue sample in each subject was calculated and plotted. Circles denote individual observations, while squares with error bars represent group means with their 95% confidence intervals (CIs). CSPG4 and CHST11 were elevated 3.2 (P<0.02) and 1.8 (P=0.034) fold, respectively in tumor-containing samples over normal samples.

FIG. 7. Enzymatic Removal of Surface CS-GAGs Reduced Lung Colonization of 4T1 Cells.

4T1 cells were treated with chondroitinase ABC or buffer and injected into the tail vein of BALB/c mice (10 per group). Mice were sacrificed 25 days later and the number of metastases to the lung was measured by clonogenic assay and expressed as “Lung Metastases”. Boxes show medians and quartiles while whiskers show ranges; plus signs indicate means. P=0.0002 by Wilcoxon rank-sum test.

FIG. 8. CHST11 expression is associated with tumor progression. A) 7 DCIS and 7 IDC tumor specimens were compared for the expression of CHST11. The expression data were downloaded from Oncomine database and analyzed as indicated. P=0.002 using two-sided t-test comparison done by Oncomine B) A total of 158 specimens were originally used for detection of gene expression in specimens with various grades. According to the data from Oncomine database 21 (13%) specimens had no value and therefore removed from the analysis. 19 of the remaining samples were diagnosed with grade 1, 49 with grade 2 and 69 with grade 3. Specimens with log 2 median-centered ratio of less or equal to “0” were classified as low expression and the rest with the expression levels above “0” were classified as high expression. A chi-square test for trend was run and the P value is shown.

FIG. 9. The Genomic Configuration of the CHST11 Gene and Methylation of CPG Island in MCF7 and MDA-MB-231 Cells.

A) Exon 1 from NCBI NM_{—}018413.5 is shown (0 to +616) as is the CpG island (between −660 and +2100). The horizontal arrows indicate bisulfite genomic sequencing primers for semi-nested PCR. Primer A is the outside forward primer (Table 4), primer B is the nested forward primer (position +1490 & Table 4), and primer C is the reverse primer (position +1750 & Table 4). The genomic sequence (pre-bisulfite) between the B and C primers is shown below the map and B and C primer locations are underlined. CpG islands are in bold and underlined. The scale is in base pairs. B) Sections of bisulfite genomic sequencing results showing part of the CHST11 CpG island. Top: DNA of MCF-7 cells. In this top sequence CpG sites show prominent cytosine (C) peaks in the electropherogram and show C in the sequence because methylated cytosines are not changed to thymines (Ts) in the bisulfite reaction. Bottom: DNA of MDA-MB-231 cells. In this section CpG sites appear as TpG sites and are in the same sequence positions as the top CpGs. In the electropherogram for the MDA-MB-231 DNA most CpG sites have prominent T peaks because unmethylated Cs are changed to uracils in the bisulfite reaction. The Cs in MCF7 that are changed to T in MDA-MB-231 are underlined and bolded. C) Quantification of methylation levels of the CpG island in MCF7 and MDA-MB-231 cells. Methylation levels were averaged over 10 CpGs and 18 preparations for MCF7 and 16 preparations for MDA-MB-231 cells. Means and SEM are shown. Data were arcsin transformed and subjected to Mann-Whitney test to compare means.

FIG. 10. 5AzadC Treatment Increases CHST11 Gene Expression in MCF7 Cells.

Gene expression was assayed by real time RT-PCR (A) and flow cytometry (B). MCF7 cells were grown in the presence of various concentrations of 5AzadC for 5 days and then cells were harvested for RNA purification or staining with anti-CS-A mAb 2H6. A) CHST11 mRNA levels are shown relative to mRNA levels of cells grown in medium only. GAPDH was used as the house keeping gene to normalize mRNA-based expression data using the delta delta CT method. Data were log transformed and subjected to one way ANOVA with post-hoc Tukey's analysis. B) Cells were harvested and then stained with anti-CS-A mAb 2H6. Binding was analyzed by flow cytometry. Mean fluorescent intensities of two independent experiments were log transformed and analyzed by ANOVA and post-hoc comparison. ns, not significant as compared to control (0 μM 5AzadC). *, **, and ***, significantly different compared to control at P≦0.05, P≦0.01, and P≦0.001, respectively.

FIG. 11. CHST11 Expression and Methylation Status of its CpG Island in Breast Cancer Cell Lines.

A) The expression of CHST11 was measured in MCF7, T-47D, ZR-75-1 and MDA-MB-231 cells by qRT-PCR and normalized using 18S. Data were analyzed by one-way ANOVA and post hoc analysis using data from 3 independent experiments. Means, standard deviations, statistically significant differences and P values are shown. B) through E) Single nucleotide resolution of DNA methylation in the CHST11 CpG island. DNA methylation was assessed by RRBS (Meissner, Mikkelsen et al. 2008). The peaks denote Cs covered with at least 10 reads. Methylation level percentage was calculated from the number of Cs after bisulfite treatment divided by the sum of Cs (methylated sites) and Ts (unmethylated sites). There is a total of 186 CpG dinucleotides in the CpG island, of which at least 75% were accessible to RRBS with a minimum coverage of 10 (average coverage at least 40). The x axis shows CHST11 in the coordinates relative to the transcription start site. The first exon is from position 1 to about position 600. Panels B, C, D, and E are respectively MCF-7, T-47D, ZR-75-1, and MDA-MB-231 cell lines.

FIG. 12. Expression Estimate and Single Nucleotide Resolution of DNA Methylation in the CHST11 CpG Island.

CHST11 gene expression and methylation of ER-positive (BT-474) and two ER-negative cell lines with epithelial appearance (MDA-MB-468 and BT-20), as described in the legend to FIG. 4. EE, expression estimate, EE is an estimate of the number of transcribed copies of CHST11 (C_{CHST11}) in the region covered by primers used for qRT-PCR performed by analyzing the digital expression of mRNA-seq data as described in Materials and Methods. LogEE is shown to be consistent with qRT-PCR data.

FIG. 13. CHST11 Expression is Associated with Basal-Like Molecular Subtype and ER-Negative Diagnosis.

A) CHST11 gene expression was evaluated in a panel of breast cancer cell lines (13 Luminal, 16 Her-2-amplified and 21 basal-like cell lines). The log-transformed normalized expression values was downloaded from Oncomine database and analyzed by one way ANOVA and Tukey's post hoc comparisons. P values comparing Her-2-amplified and basal-like subtypes with Luminal subtype are shown. B) CHST11 expression data in 158 invasive breast carcinomas were downloaded from Oncomine database and analyzed. The data of 7 specimens with no value were removed. 70 of the remainings were diagnosed as ER-negative and 81 as ER-positive. The Mann-Whitney test was performed to compare CHST11 expression in ER-negative versus ER-positive specimens and P-value is shown.

FIG. 14. CHST11 Expression Levels are Significantly Higher in Invasive Breast Carcinoma Vs. Normal Breast.

Expression levels in 61 normal breast and 76 invasive breast adenocarcinoma samples were analyzed. The dataset consists of Level 2 (processed) data from the TCGA data portal.

FIG. 15. CHST11 is Overexpressed in Cutaneous Melanoma Compared to Normal Skin.

The dataset was 14 cutaneous melanoma samples and 4 normal skin.

FIG. 16. CHST11 is Overexpressed in Oligodendroglioma Compared to Normal Brain.

The data set was 23 normal brain samples and 50 oligodendroglioma samples.

FIG. 17. CHST11 Expression is Associated with Basal-Like Molecular Subtype of Breast Cancer.

CHST11 gene expression was evaluated in a panel of breast cancer cell lines (13 Luminal, 16 Her-2-amplified and 21 basal-like cell lines). The log-transformed normalized expression values was downloaded from Oncomine database and analyzed by one way ANOVA and Tukey's post hoc comparisons. P values comparing Her-2-amplified and basal-like subtypes with Luminal subtype are shown.

FIG. 18. CHST11 Expression is Associated with ER-Negative Breast Cancer.

CHST11 expression data in 158 invasive breast carcinomas were downloaded from Oncomine database and analyzed. The data of 7 specimens with no value were removed. 70 of the remainings were diagnosed as ER-negative and 81 as ER-positive. The Mann-Whitney test was performed to compare CHST11 expression in ER-negative versus ER-positive specimens and P-value is shown.

### DETAILED DESCRIPTION

### Abbreviations

18S, 18S ribosomal RNA; ANOVA, Analysis of Variance; CS, Chondroitin Sulfate; CS-GAGs, Chondroitin Sulfate Glycosaminoglycans; CS/DS, Chondroitin Sulfate/Dermatan Sulfate; CS/DS GAGs, Chondroitin Sulfate/Dermatan Sulfate Glycosaminoglycans; CHST3, Carbohydrate (chondroitin 6) sulfotransferase 3; CHST7, Carbohydrate (N-acetylglucosamine 6-O) sulfotransferase 7; CHST11, Carbohydrate (Chondroitin 4) Sulfotransferase 11; CHST12, Carbohydrate (Chondroitin 4) Sulfotransferase 12; CHST13; Carbohydrate (Chondroitin 4) Sulfotransferase 13; CHST14, Carbohydrate (N-acetylgalactosamine 4-O) Sulfotransferase 14; CHST15, Carbohydrate (N-acetylgalactosamine 4-sulfate 6-O) Sulfotransferase 15; CSPG4, Chondroitin Sulfate Proteoglycan 4; CS-A, Chondroitin Sulfate A unit; CS-E, Chondroitin Sulfate E unit; CSPG4, Chondroitin Sulfate Proteoglycan 4; DS, Dermatan Sulfate; DS4S-1, Dermatan 4-sulfotransferase 1; ER1, Estrogen Receptor 1; GAGs, Glycosaminoglycans; GalNAc, N-acetyl-D-galactosamine; GalNAc4S-6ST, N-acetylgalactosamine 4-sulfate 6-O-sulfotransferase; GAPDH, Glyceraldehyde-3-phosphate dehydrogenase; GlcNAc, N-acetyl-D-glucosamine; GlcA, Glucuronic acid; IdoA, Iduronic acid; mAb, monoclonal Antibody; NPR-1, Neuropilin-1; PG, Proteoglycan; qRT-PCR, Quantitative Real-Time Polymerase Chain Reaction; SDC-1, Syndecan-1; SDC-4, Syndecan-4; siRNA, short interfering RNA; UAMS, University of Arkansas for Medical Sciences.

### DESCRIPTION

It is shown here in Example 1 that the CHST11 gene is highly expressed in aggressive breast cancer cells but significantly less so in less aggressive breast cancer cell lines. Furthermore, a positive correlation was observed between the expression levels of CHST11 and P-selectin binding to cells, which is a mechanism important for metastasis. Blocking the expression of CHST11 with siRNA inhibited CS-A expression and P-selectin binding to MDA-MB-231 cells. The carrier proteoglycan CSPG4 was also highly expressed on the aggressive breast cancer cell lines and contributed to the P-selectin binding and CS-A expression. In addition, CSPG4 and CHST11 were over-expressed in tumor-containing clinical tissue specimens compared with normal tissues. It was also shown that enzymatic removal of tumor-cell surface CS-GAGs significantly inhibited lung colonization of the 4T1 murine mammary cell line, which shows the importance of the CS-GAGs for metastasis and probably explains why CHST11 overexpression correlates with more aggressive cancer cell lines.

In Example 2, the inventors show that CHST11 overexpression in cancer tissues correlates with hypomethylation of the CHST11 gene. Likewise, the inventors have found that overexpression of the CSPG4 gene in cancer tissues correlates with hypomethylation of the CSP4 gene. This means that overexpression of the CHST11 and CSPG4 genes can be inferred in a tumor when the tumor tissue has these genes hypomethylated.

Direct measurement of gene expression requires living tumor cells or well-prepared frozen samples, but paraffin-embedded or catalogued stained tissues, can be used for DNA samples that will show whether these genes are hypomethylated or hypermethylated. Thus, it is easier to determine methylation state of these genes than to directly measure overexpression, and archived tumor tissue samples can be used to determine methylation status that cannot be used for measurement of gene overexpression.

The inventors have found that the expression of CHST11 and the methylation of its DNA sequence can be used for diagnostic/prognostic and predictive purposes. A combination of the expression of two sulfotransferases like CHST3 and CHST11 can be used. A combination of a CHST11 and structural proteoglycan genes such as CSPG4, syndecan-1, syndecan-4, or neuropilin-1 can be used.

But most preferably, expression level and/or methylation state of CHST11 in cancer tissue is used to provide a prognosis. This can be combined, most preferably with determination of overexpression and/or methylation state of CSPG4 in the cancer tissue.

Detection of high expression of CHST11 if combined with hypomethylation of a particular region of the CHST11 gene identify highly aggressive tumors (triple negative and basal-like). Detection of low expression due to hypermethylation will indicate less aggressive tumor cells (Estrogen receptor positive, Luminal-like). Low expression and a hypomethylated sequence indicate normal cells.

This can be used in primary tumor for prognostic purposes.

We have obtained data analyzed herein showing that CHST11 is overexpressed in many types of cancers.

The method can be used in circulating tumor cells (CTC) enriched and isolated from blood for predictive purposes.

The expression levels can also be used for detection and measuring of CTCs in blood.

The methylated DNA can be detected in plasma or serum samples for diagnostic and prognostic purposes.

The high expression of CHST11 can be combined with low expression of CHST3 for more accurate detection of cancer cells.

The high expression of CHST11 can be combined with high expression of CSPG4 for more accurate prognostic and predictive detection.

In particular embodiments of the methods of determining a prognosis of a cancer in a human, the cancer tissue sample tested for gene expression level or gene methylation status may be a blood sample, an enriched circulating tumor cell sample, a living or frozen tumor biopsy sample, or an archived paraffin-embedded tumor biopsy sample, for instance a fixed and stained paraffin-embedded slide preparation.

The cancer may be any cancer. In specific embodiments, it is breast cancer, an epithelial cancer generally, osteosarcoma, brain cancer, or a blood cancer (e.g., lymphoma, leukemia, or myeloma). In other specific embodiments, it may be melanoma, cervical cancer, esophagus cancer, head and neck cancer, or pancreatic cancer.

In the samples we have tested, particularly in the region of base pairs 1490-1850 (SEQ ID NO:37) (numbered from the transcription start site) of the CHST11 gene (NCBI accession number NG_{—}029810.1), methylation in tumor samples at most CpG sites is either above 90% or below 10%. Thus, it is clear whether a sample is hypomethylated or hypermethylated.

Expression levels of cancer tissue samples can be assayed by real time PCR. The levels can be compared a low expression sample such as MCF-7 cells or a high expression sample, such as MDA-MB-231 cells.

For analysis of circulating epithelial tumor cells, the cells can be enriched by use of the ROSETTESEP® Human Circulating Epithelial Tumor Cell Enrichment Cocktail (Stem Cell Technologies, Vancouver, Canada). The ROSETTESEP™ Human Circulating Epithelial Tumor Cell Cocktail is designed to enrich circulating epithelial tumor cells from fresh whole blood by negative selection. Unwanted cells are targeted for removal with Tetrameric Antibody Complexes recognizing CD2, CD16, CD19, CD36, CD38, CD45, CD66b and glycophorin A on red blood cells (RBCs). When centrifuged over a buoyant density medium such as Ficoll, the unwanted cells pellet along with the RBCs. The purified tumor cells are present as a highly enriched population at the interface between the plasma and the buoyant density medium.

Thus, again, one embodiment A method of determining a prognosis of a cancer in a human comprising: (1) determining expression level of CHST11 in a cancer tissue sample or determining methylation status of CHST11 gene in a cancer tissue sample; and/or (2) determining expression level of CSPG4 in a cancer tissue sample or determining methylation status of CSPG4 gene in a cancer tissue sample. For both CHST11 and CSPG4, high expression is indicative of more aggressive tumors and tumors more likely to metastasize. And for CHST11 and CSPG4, hypomethylation of the genes in tumor samples correlates with high expression of the genes.

Expression levels are preferably determined by real-time PCR amplifying mRNA from living tumor biopsy tissues or from circulating tumor cells. Where antibodies are available to the proteins, expression levels can also be determined by measuring levels of the CHST11 product or CSPG4 protein.

Having a prognosis that indicates a less aggressive tumor type or a more aggressive tumor type has benefits for both the treating physician and the patient. It can help inform decisions about how aggressively to treat the cancer. It also allows patients to have a better idea of how much time they may have left to live or how likely they are to survive the cancer, and thus to plan their lives, and also helps them to decide which treatments they want to undergo.

The more aggressive tumor types indicated by overexpression of CHST11 have the traits of appearing more mesenchymal and less epithelial. They are more prone to invade the basement membrane, and thus more invasive. And they are more prone to metastasize.

In some cases, sensitivity or resistance to certain drugs can also be indicated by expression levels of CHST11 and CSPG4. CHST11 high expressing cells are more sensitive to daunorubicin (Gyorffy B, et al. Oncogene. 2005 Nov. 17; 24(51):7542-51.), to Selumetinib (an inhibitor of MEK and MAPK/ERK kinases; Garnett, M J. et al Nature 2012 Mar. 28; 483(7391):570-5), and CHIR-265 (an inhibitor of RAF and VEGFR; Barretina, J. et al Nature. 2012 Mar. 28; 483(7391):603-7.), and less sensitive to cisplatin (Gyorffy, B. et al. Int J. Cancer. 2006 Apr. 1; 118(7):1699-712) and trastuzumab (Neve, R M. Et al, Cancer Cell. 2006 December; 10(6):515-27).

The CHST11 gene sequence is genbank nucleotide accession number NG_{—}029810.1.

The transcription start stie in this gene is at nucleotide 5001. This is position 1 in FIG. 9A below. The area CpG isand for this gene is from approximately −660 to 2100, numbered relative to the transcription start. The area of greatest difference in methylation status is positions 1490 to 1850, shown in FIG. 9A. This is SEQ ID NO:37 and corresponds to positions 6490 to 6850 of accession number NG_{—}029810.1.

The cDNA for the human CSPG4 gene is genbank accession number NM_{—}001897.4. The gene is located on chromosome 15, accession number NC_{—}000015.9. The transcription start is at nucleotide 76005190. Exon 1 begins at nucleotide 76005097. This gene sequence can be used to test for methylation.

### EXAMPLES

### Example 1

### Chondroitin Sulfates Play a Major Role in Breast Cancer Metastasis: a Role for CSPG4 and CHST11 Gene Expression in Forming Surface P-Selectin Ligands in Aggressive Breast Cancer Cells

Summary:

We have previously demonstrated that chondroitin sulfate glycosaminoglycans (CS-GAGs) on breast cancer cells function as P-selectin ligands. This study was performed to identify the carrier proteoglycan (PG) and the sulfotransferase gene involved in synthesis of the surface P-selectin-reactive CS-GAGs in human breast cancer cells with high metastatic capacity, as well as to determine a direct role for CS-GAGs in metastatic spread.

### Methods:

Quantitative real-time PCR (qRT-PCR) and flow cytometry assays were used to detect the expression of genes involved in the sulfation and presentation of chondroitin in several human breast cancer cell lines. Transient transfection of the human breast cancer cell line MDA-MB-231 with the siRNAs for carbohydrate (chondroitin 4) sulfotransferase-11 (CHST11) and chondroitin sulfate proteoglycan 4 (CSPG4) was used to investigate the involvement of these genes in expression of surface P-selectin ligands. The expression of CSPG4 and CHST11 in 15 primary invasive breast cancer clinical specimens was assessed by qRT-PCR. The role of CS-GAGs in metastasis was tested using the 4T1 murine mammary cell line (10 mice per group).

### Results

The CHST11 gene was highly expressed in aggressive breast cancer cells but significantly less so in less aggressive breast cancer cell lines. A positive correlation was observed between the expression levels of CHST11 and P-selectin binding to cells (P<0.0001). Blocking the expression of CHST11 with siRNA inhibited CS-A expression and P-selectin binding to MDA-MB-231 cells. The carrier proteoglycan CSPG4 was highly expressed on the aggressive breast cancer cell lines and contributed to the P-selectin binding and CS-A expression. In addition, CSPG4 and CHST11 were over-expressed in tumor-containing clinical tissue specimens compared with normal tissues. Enzymatic removal of tumor-cell surface CS-GAGs significantly inhibited lung colonization of the 4T1 murine mammary cell line (P=0.0002).

### Conclusions

Cell surface P-selectin binding depends on CHST11 gene expression. CSPG4 serves as a P-selectin ligand through its CS chain and participates in P-selectin binding to the highly metastatic breast cancer cells. Removal of CS-GAGs greatly reduces metastatic lung colonization by 4T1 cells. The data strongly indicate that CS-GAGs and their biosynthetic pathways are promising targets for the development of anti-metastatic therapies.

### Introduction

Tumor-associated glycans play a significant role in promoting aggressive and metastatic behavior of malignant cells [1-5], participating in cell-cell and cell-extracellular matrix interactions that promote tumor cell adhesion and migration. Among glycans that play a critical role in stromal tumor cell interactions are glycosaminoglycans (GAGs) attached to proteoglycans (PGs). Altered production levels of PGs and structural changes in their GAGs are reported in many neoplastic tissues [6-10]. GAGs are polysaccharide chains covalently attached to protein cores that together comprise PGs [6, 11] and based on the prevalence of GAG chains, chondroitin sulfate (CS)/dermatan sulfate (DS) PGs (CS/DS-PGs), heparan sulfate PGs and keratan sulfate PGs have been described [12]. Increased production of CS/DS-GAGs is found in transformed fibroblasts and mammary carcinoma cells [8, 13, 14] and it has been shown that these polysaccharides contribute to fibrosarcoma cell proliferation, adhesion and migration [15].

Several studies have disclosed the critical involvement of P-selectin in the facilitation of blood borne metastases [16-18]. P-selectin/ligand interaction often requires sialylated and fucosylated carbohydrate such as sialyl Lewis X and sialyl Lewis A [19]; however, P-selectin also binds to heparan sulfate, certain sulfated glycolipids and CS/DS-GAGs [20-23]. In previous studies we found that CS/DS-GAGs are expressed on the cell surface of murine and human breast cancer cell lines with high metastatic capacity and that they play a major role in P-selectin binding and P-selectin-mediated adhesion of cancer cells to platelets and endothelial cells [24]. However, variation in the abundance and function of CS/DS relative to tumor cell phenotypic properties and P-selectin binding are not well defined. It is likely that P-selectin binding to tumor cells and the functional consequences of such binding are dependent on which sulfotransferases define the relevant CS/DS and which core proteins carry the CS polysaccharide.

CS/DS expression is controlled by many enzymes in a complex biosynthetic pathway and this leads to considerable variation in structure and function. The chondroitin backbone of CS/DS-GAGs consists of repetitive disaccharide units containing D-glucuronic acid (GlcA) and N-acetyl-D-galactosamine (GalNAc) residues, or varying proportions of L-iduronic acid (IdoA) in place of GlcA [25, 26]. Major structural variability of the CS/DS chains is due to the sulfation positions in repeating disaccharide units by the site-specific activities of sulfotransferases that produce the variants CS-A, CS-B (dermatan sulfate, DS), CS-C, CS-D and CS-E [26, 27]. CHST3, CHST7, CHST11, CHST12, CHST13, CHST14 and CHST15 are the enzymes that sulfate the GalNAc residues of chondroitin polymers. The expression levels of these enzymes are hypothesized to affect the production of P-selectin ligands. CHST11 and CHST13 share specificity, as they mediate 4-0 sulfation of chondroitin [28-30]. CHST12 and CHST14 mediate mostly 4-0 sulfation of DS units [30, 31]. CHST3 and CHST7 share specificity for 6-0 sulfation of chondroitin [32, 33]. CHST15 transfers sulfate to the carbon-6 of an already 4-0 sulfated GalNAc residue producing oversulfated CS-E [34]. The relationship between the relative expression of these sulfotransferases with the expression of P-selectin-reactive CS/DS-GAGs has not been reported and is addressed in this paper.

The variation, abundance and function of CS/DS-GAGs are also affected by the expression of the PG core protein presenting them. Syndecan-1 (SDC-1), syndecan-4 (SDC-4), neuropilin-1 (NRP-1) and CSPG4 are considered major pro-malignancy membrane proteins capable of carrying CS chains [35-39]. Among these PGs, CSPG4 exclusively carries CS chains [40, 41]. CSPG4 is a human homolog of Rat NG2, which is also known as High Molecular Weight Melanoma Associated Antigen and Melanoma Chondroitin Sulfate Proteoglycan [40, 42, 43]. This tumor-associated cell surface PG potentiates cell motility [44-46]. CSPG4 is also linked to cancer stem cells via signaling mechanisms [39, 45]. Therefore, studying whether this PG interacts directly with P-selectin is of particular interest.

Depending on CSPG4's relative expression levels, this PG might be a major core protein presenting CS-GAGs on an aggressive subset of tumor cells, interacting with P-selectin. In the current study we investigated whether CSPG4, via its CS/DS chain can serve as a P-selectin ligand and whether expression of specific chondroitin sulfotransferases contributes to P-selectin interaction with cells. We further examined the involvement of CS-GAG chains in lung colonization by the 4T1 mammary cell line with and without enzymatic removal of CS-GAGs in a murine experimental metastasis model. Our data show for the first time that P-selectin can bind to tumor cells via CSPG4 and that CHST11 expression is linked to P-selectin-reactive cell surface CS/DS-GAGs. The results directly link CS/DS-GAGs to the metastatic spread of breast cancer. These findings have significant implications for understanding mechanisms of breast cancer metastasis, paving the way towards developing alternative strategies to treat and prevent metastasis.

### Materials and Methods

### Reagents

Anti-CS-A mAb 2H6 was from Associates of Cape Cod/Seikagaku America (Falmouth, Mass., USA), anti-CSPG4 mAb 225.28 was made as described [47, 48], and recombinant human P-selectin/Fc (human IgG) was from R&D Systems (Minneapolis, Minn., USA). Fluorescence-conjugated anti-human IgG, anti-mouse IgM and chondroitinase ABC were from Sigma (St. Louis, Mo., USA). R-Phycoerythrin-conjugated polyclonal goat anti-mouse F(ab′)₂ fragment was from Dako North America, Inc. (Carpinteria, Calif., USA). DNA primers were from Integrated DNA Technologies (IDT, Coralville, Iowa, USA). Real-time PCR reagents were from Applied Biosystems (Foster City, Calif., USA). Pre-designed siRNA sequences were from Ambion (Austin, Tex., USA) and Santa Cruz Biotechnology Inc. (Santa Cruz, Calif., USA). siPORT™ NeoFX™ Transfection Agent was from Ambion. TRIzol reagent was from Invitrogen (Carlsbad, Calif., USA).

### Cell Lines and Tissue Specimens

Human breast cancer MCF7, MDA-MB-231, MDA-MB-468 cell lines and the murine 4T1 cell line were from ATCC (Manassas, Va., USA). Human MDA-MET cells were selected in vivo for their bone colonizing phenotype [49]. 4T1 cells were used within 10 passages and less than six months after receipt. We confirmed cell line identities by the Human Cell Line Authentication test (Genetica DNA Laboratories, Inc. Cincinnati, Ohio, USA). The melanoma cell lines M14 and M14-CSPG4, stably transfected to express CSPG4, were used as homologous CSPG4-non-expressing and expressing cell lines [50] and were characterized by real-time PCR for CSPG4 expression. Cells were cultured in a base medium supplemented with 10% heat-inactivated fetal bovine serum (Life Technologies, Carlsbad, Calif., USA), 50 units/mL penicillin, and 50 μg/mL streptomycin. Base medium for MDA-MB-231, MDA-MET, MDA-MB-468, and 4T1 was DMEM (Fisher Scientific, Pittsburgh, Pa., USA). For MCF7 base medium was MEM (Fisher Scientific) supplemented with 0.1 mM non-essential amino acids, 1 mM sodium pyruvate, and 0.01 mg/ml insulin (Invitrogen). For M14 and M14/CSPG4 RPMI 1640 medium (Fisher Scientific) was used with the addition of 500 μg/ml G418 (Invitrogen). Cells are checked every six months to be free from Mycoplasma contamination using the MycoAlert® Mycoplasma Detection Kit (Lonza Rockland Inc., Rockland, Me., USA).

De-identified frozen specimens from 15 female breast cancer patients diagnosed with invasive ductal carcinoma were provided by the University of Arkansas for Medical Sciences (UAMS) Tissue Procurement Facility. Specimens were matched for each donor to have both tumor-free and tumor-containing breast tissues. According to the policy of the Tissue Procurement Facility, patients signed consent forms to donate excess specimen tissue and to allow use of related pathological data for research purposes, which were approved by the UAMS Institutional Review Board. For this study, an active human tissue use protocol approved by the UAMS Institutional Review Board was used. In this protocol, informed consent was waived due to the use of de-identified specimens with no link to patient identifiers.

Total RNA Isolation and qRT-PCR

Total RNA was isolated from cultured cells and tumor tissues using TRIzol reagent, following the manufacturer's instructions. The quantity and quality of the isolated RNA was determined by Agilent 2100 Bioanalyzer (Palo Alto, Calif., USA). One μg of total RNA was reverse-transcribed using random-hexamer primers with TaqMan Reverse Transcription Reagents (Applied Biosystems). Reverse-transcribed RNA was amplified with SYBR Green PCR Master Mix (Applied Biosystems) plus 0.3 μM of gene-specific upstream and downstream primers during 40 cycles on an Applied Biosystems 7500 Fast Realtime cycler. Data were analyzed by absolute and relative quantification. In absolute quantification, data were expressed in relation to 18S RNA, where the standard curves were generated using pooled RNA from the samples assayed. In relative quantification, the 2 (^{-Delta Delta C(T)}) method was used to assess the target transcript in a treatment group relative to that of an untreated control group using expression of an internal control (reference gene) to normalize data [51]. Expression of GAPDH and β-actin was used as internal control. Each cycle consisted of denaturation at 95° C. for 15 s, and annealing and extension at 60° C. for 60 s. The primer sequences are shown in Table 1.

### Flow Cytometry

Binding of the recombinant human P-selectin/Fc molecule, anti-CS-A (2H6) and anti-CSPG4 (225.28) mAb to cells was determined using flow cytometry as previously described [24]. Briefly, cells were incubated with mAb or P-selectin/Fc, washed and stained with FITC-conjugated anti-mouse IgM or anti-human IgG prior to binding detection by flow cytometry. R-phycoerythrin-conjugated polyclonal goat anti-mouse F(ab′)₂ was used as secondary for detection of anti-CSPG4 binding.

Switching Off Gene Expression with siRNA

Three pre-designed siRNA sequences for CHST11 (Ambion) were used (Table 1). NG2 siRNA (sc-40771) from Santa Cruz Biotechnology, Inc. was used for inhibition of CSPG4. These sequences were transfected into cells growing in tissue culture using the siPORT™ NeoFX™ Transfection Agent, and mRNA levels were determined 48 hours later by real-time PCR. Expression of GAPDH was used as reference control. Reactivity of anti-CS-A mAb 2116 and human recombinant P-selectin was assessed 144 hours after siRNA transfection. Transfection with GAPDH siRNA (Ambion) was used as control.

TABLE 1
Primer and siRNA sequences used in this study

| | Sequence |
|---|---|
| Primer | |
| 18S forward | 5′ TTCGAACGTCTGCCCTATCAA 3′ (SEQ ID NO: 1) |
| 18S reverse | 5′ ATGGTAGGCACGGCGACTA 3′ (SEQ ID NO: 2) |
| β-actin forward | 5′ GACTTCGAGCAAGAGATGGCCAC 3′ (SEQ ID NO: 3) |
| β-actin reverse | 5′ CAATGCCAGGGTACATGGTGGTG 3′ (SEQ ID NO: 4) |
| CHST3 forward | 5′ GACTTTGTGCACAGCCTGAA 3′ (SEQ ID NO: 5) |
| CHST3 reverse | 5′ AGAGCTTGGGGAATCTGCTT 3′ (SEQ ID NO: 6) |
| CHST7 forward | 5′ GGGGCAATCTGTCACACTCT 3′ (SEQ ID N07) |
| CHST7 reverse | 5′ AATTGCACAGCAGTIGTTGG 3′ (SEQ ID NO: 8) |
| CHST11 forward | 5′ TCCCTTTGGTGTGGACATCT 3′ (SEQ ID NO: 9) |
| CHST11 reverse | 5′ CACGTGTCTGTCACCTGGTC 3′ (SEQ ID NO: 10) |
| CHST12 forward | 5′ GCACACGTCCTTCTCTAGGC 3′ (SEQ ID NO: 11) |
| CHST12 reverse | 5′ AAACTCGTCGACATCGGAGT 3′ (SEQ ID NO: 12) |
| CHST13 forward | 5′ CCGGCATTTGGAAACAGAG 3′ (SEQ ID NO: 13) |
| CHST13 reverse | 5′ GGGTCCTGATCCAGGTCATA 3′ (SEQ ID NO: 14) |
| CHST14 forward | 5′ TCTGATCCCCCATTTATCCA 3′ (SEQ ID NO: 15) |
| CHST14 reverse | 5′ GGAGCAGAAAGGCACAAAAG 3′ (SEQ ID NO: 16) |
| CHST15 forward | 5′ TGAGTTCACGACCAGACAGC 3′ (SEQ ID NO: 17) |
| CHST15 reverse | 5′ CGTACCAACAGGGGCTCTTA 3′ (SEQ ID NO: 18) |
| CSPG4 forward | 5′ CACACAGAGGAACCCTCGAT 3′ (SEQ ID NO: 19) |
| CSPG4 reverse | 5′ CTTCAGCGAGAGGAGCACTT 3′ (SEQ ID NO: 20) |
| ER1 forward | 5′ AGGTGGGATACGAAAAGACCG 3′ (SEQ ID NO: 21) |
| ER1 reverse | 5′ AAGGTTGGCAGCTCTCATGTC 3′ (SEQ ID NO: 22) |
| GAPDH forward | 5′ ACAGTCAGCCGCATCTTCTT 3′ (SEQ ID NO: 23) |
| GAPDH reverse | 5′ ACGACCAAATCCGTTGACTC 3′ (SEQ ID NO: 24) |
| NRP-1 forward | 5′ CAAGGTGTTCATGAGGAAGTTCAA 3′ (SEQ ID NO: 25) |
| NRP-1 reverse | 5′ CCGCAGCTCAGGTGTATCATAGT 3′ (SEQ ID NO: 26) |
| SDC-1 forward | 5′ GCTCTGGGGATGACTCTGAC 3′ (SEQ ID NO: 27) |
| SDC-1 reverse | 5′ GGTCTGCTGTGACAAGGTGA 3′ (SEQ ID NO: 28) |
| SDC-4 forward | 5′ GTCTGGCTCTGGAGATCTGG 3′ (SEQ ID NO: 29) |
| SDC-4 reverse | 5′ CACCAAGGGATGGACAACTT 3′ (SEQ ID NO: 30) |
| siRNA | |
| #31 | UCAGUAGUUCUCGUAGACUtt (SEQ ID NO: 31) |
| #32 | UUCUGGGUGAACUUGUUGCgg (SEQ ID NO: 32) |
| #33 | AUCGAGUUUGUAGACUUCGta (SEQ ID NO: 33) |

18S, 18S ribosomal RNA;
CHST3, Carbohydrate (chondroitin 6) sulfotransferase 3;
CHST7, Carbohydrate (N-acetylglucosamine 6-O) sulfotransferase 7;
CHST11, Carbohydrate (Chondroitin 4) Sulfotransferase 11;
CHST12, Carbohydrate (Chondroitin 4) Sulfotransferase 12;
CHST13, Carbohydrate (Chondroitin 4) Sulfotransferase 13;
CHST14, Carbohydrate (N-acetylgalactosamine 4-O) Sulfotransferase 14;
CHST15, Carbohydrate (N-acetylgalactosamine 4-sulfate 6-O) Sulfotransferase 15;
CSPG4, Chondroitin Sulfate Proteoglycan 4;
ER1, Estrogen Receptor 1;
GAPDH, Glyceraldehyde-3-phosphate dehydrogenase;
NPR-1, Neuropilin-1;
SDC-1, Syndecan-1;
SDC-4, Syndecan-4;
siRNA, short interfering RNA.

### Mice and Tumor Models

BALB/c female mice (six to eight weeks old) were from Harlan Laboratories (Indianapolis, Ind., USA). We used 4T1 cells in an experimental metastasis model [24]. Cells were treated with chondroitinase ABC in HBSS buffer with protease inhibitors [24], or with the buffer and protease inhibitor alone (no chondroitinase ABC treatment) before inoculation through the tail vein. Each mouse (10 mice per group) received 2×10⁴ 4T1 cells. Mice were sacrificed 25 days after tumor cell injection and lungs were harvested to determine clonogenic cells by growing cells in medium containing 6-thioguanine [52]. Animal studies have been reviewed and approved by the Institutional Care and Use Committee of UAMS.

### Statistical Analysis

For comparison of gene expression between cell lines, the raw amount for each mRNA was normalized to the control mRNA (18S) amount and then log transformed, and analyzed via one-way ANOVA with Tukey's post-hoc procedure. For the tissue comparisons of gene expression in patient samples, the tumor/normal expression ratios for each patient were log-transformed and subjected to one-sample t-tests. For siRNA effects on relative mRNA levels mean fluorescence intensities of antibody binding were log transformed and analyzed via ANOVA and Tukey's post-hoc procedure. Associations between mean fluorescence intensities of P-selectin binding and quantities of sulfotransferase transcripts were characterized using Pearson correlation tests on log-transformed data. In order to check the validity of assumptions for running Pearson's test, Spearman correlation analysis was also performed and when correlation coefficients were similar, assumptions were considered valid. For clonogenic assay comparisons, the number of clonogenic lung metastases was analyzed between groups by the Wilcoxon rank-sum test.

### Results

CHST11 is Overexpressed in Aggressive Human Breast Cancer Cell Lines and its Expression Correlates with P-Selectin Binding

We have shown that CS/DS-GAGs expressed on the cell surface of MDA-MB-231 and MDA-MET human breast cancer cells function as P-selectin ligand and that exogenous CS-E efficiently inhibits P-selectin binding to cells [24]. Among the major sulfotransferases able to sulfate the GlaNAC residue of the chondroitin disaccharide, CHST11, CHST12, and CHST13 are chondroitin 4-sulfotransferases, able to catalyze sulfation of chondroitin on the carbon-4 position of GalNAc sugars in disaccharide GAG units, producing primarily CS-A units (GlcAβ1-3GalNAc (4-SO₄)) [28-30]. CHST14, dermatan 4-sulfotransferase 1 (D4ST-1), is specific for 4-O sulfation of the GalNAc in CS-B (DS) units after dermatan sulfate epimerase activity and its specificity can be shared by CHST12 [30, 31]. CHST3 and CHST7 are chondroitin 6-sulfotransferases that transfer sulfate to carbon-6 position of GalNAc, competing with 4-sulfotransferases for the same substrate, producing primarily CS—C units (GlcAβ1-3GalNAc (6-SO₄)) [32, 33]. CHST15, GalNAc 4-sulfate 6-O-sulfotransferase (GalNAc4S-6ST), transfers sulfate to the carbon-6 of a 4-sulfated GalNAc, producing CS-E (GlcAβ1-3GalNAc (4-, 6-SO₄)) [34].

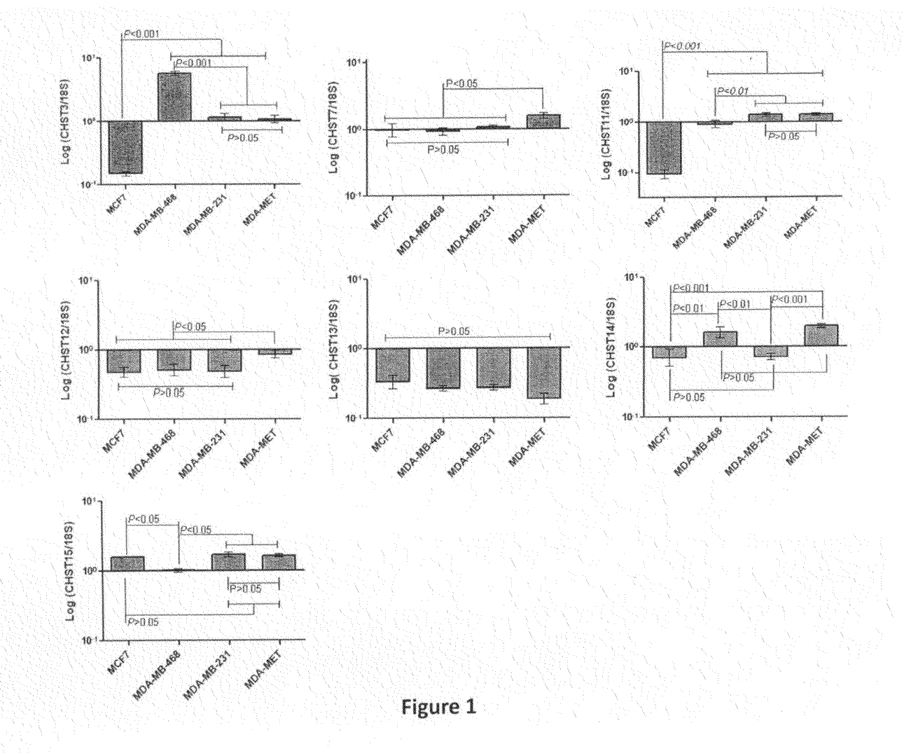

Quantitative real-time PCR was used to monitor the expression levels of these genes in several human breast cancer cell lines differing in their cancer phenotype. The human breast cancer cell lines MCF7, MDA-MB-468, MDA-MB-231, and MDA-MET represent increasing aggressiveness and metastatic capacity (MCF7<MDA-MB-468<MDA-MB-231=MDA-MET). While expression of all genes was detected in these breast cancer cells, CHST13 expression was observed to be very low with no significant differences in expression between cell lines (FIG. 1). Among the seven sulfotransferases tested, only CHST11 expression levels trended well with the metastatic potential of cells, ranging from a low in MCF7, intermediate to high in MDA-MB-468 and high in MDA-MB-231 and MDA-MET cells (FIG. 1). The expression level of CHST11 was elevated with the increase in metastatic potential of cells. Such a trend was not observed with the expression levels of other genes examined. Therefore, the data may implicate the immediate product of CHST11 expression, CS-A, in the metastatic behavior of these cells.

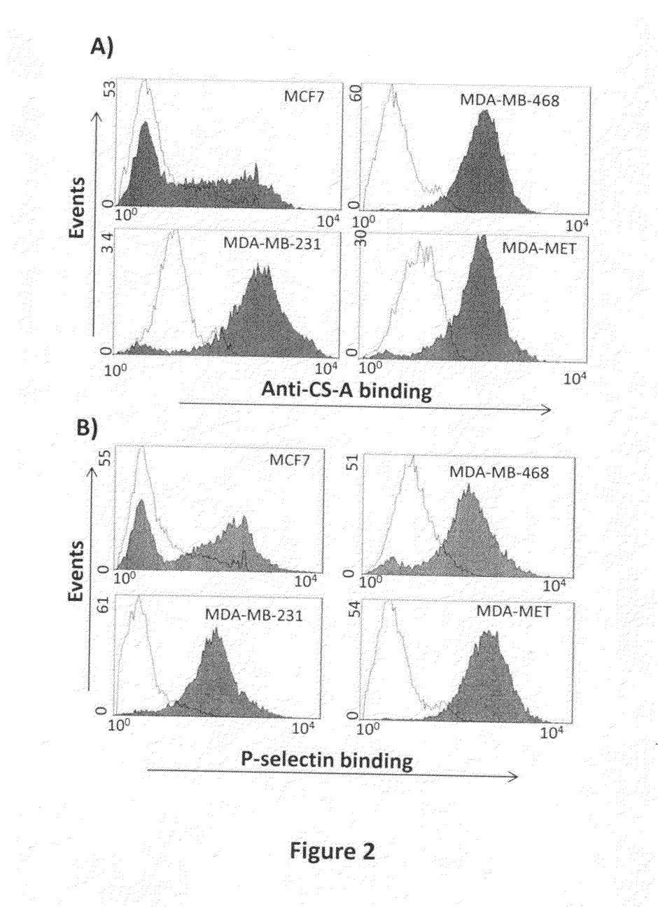

Flow cytometry analysis of the binding of the anti-CS-A mAb 2H6, consistent with qRT-PCR data, indicates that the expression of CS-A was lower in the least aggressive MCF7 cell line and high in the most aggressive MDA-MB-231 and MDA-MET cell lines (FIG. 2A). While MDA-MB-468 showed higher expression of CS-A than MCF7 cells, the CS-A expression in MDA-MB-468 was not different than its expression in MDA-MB-231 and MDA-MET. The lack of a difference in CS-A between these three cell lines could be due to the expression level of CHST15 that can turn the 4-sulfated product of CHST11 to CS-E. The expression of CHST15 was significantly lower in MDA-MB-468 than in MDA-MB-231 or MDA-MET (FIG. 1) making the conversion of some CS-A to CS-E much more likely in MDA-MB-231 and MDA-MET than in MDA-MB-468. Among the sulfotransferase genes examined, P-selectin binding to these cells correlated the best with CHST11 expression. We performed a correlation analysis of the gene expression levels and the mean fluorescence intensities of P-selectin binding among all cell lines studied and observed that only CHST11 (r=0.85, P<0.0001) showed a statistically significant correlation with P-selectin binding (Table 2). P-selectin reactivity with cell lines was comparable to anti-CSA 2H6 binding (FIG. 2A, B). These data suggest that CHST11 (a chondroitin-4-sulfotransferase) expression in these cells is associated with the production of P-selectin ligands.

TABLE 2
Correlation coefficients between P-selectin binding to cells and
the expression of the sulfotransferases studied.

| Sulfotransferase | | | P value (two- |
|---|---|---|---|
| gene | Pearson r | number | tailed) |
| CHST3 | 0.25 | 16 | 0.36 |
| CHST7 | 0.45 | 12 | 0.14 |
| CHST11 | 0.85 | 16 | <0.0001 |
| CHST12 | 0.32 | 12 | 0.30 |
| CHST13 | −0.37 | 12 | 0.24 |
| CHST14 | 0.20 | 12 | 0.52 |
| CHST15 | 0.29 | 16 | 0.31 |

Gene expression was measured by qRT-PCR and normalized using 18S transcript. Mean Fluorescence Intensities of P-selectin binding to cells was assayed by flow cytometry. Cell lines were MCF7, MDA-MB-468, MDA-MB-231, and MDA-MET. Each assay was repeated independently 3 times per cell line for CHST7, CHST12, CHST13 and CHST14 and 4 times per cell line for CHST3, CHST11, CHST15. Then the data were log transformed and subjected to correlation analysis using both Pearson (parametric) and Spearman (nonparametric) correlation tests in order to check validity of assumptions. Correlation coefficients were similar, assumptions were considered valid, and the Pearson coefficient was reported.
CHST3, Carbohydrate (chondroitin 6) sulfotransferase 3;
CHST7, Carbohydrate (N-acetylglucosamine 6-O) sulfotransferase 7;
CHST11, Carbohydrate (Chondroitin 4) Sulfotransferase 11;
CHST12, Carbohydrate (Chondroitin 4) Sulfotransferase 12;
CHST13; Carbohydrate (Chondroitin 4) Sulfotransferase 13;
CHST14, Carbohydrate (N-acetylgalactosamine 4-O) Sulfotransferase 14;
CHST15, Carbohydrate (N-acetylgalactosamine 4-sulfate 6-O) Sulfotransferase 15.

### Inhibition of CHST11 Expression Results in Inhibition of Cs-A Production and P-Selectin Binding

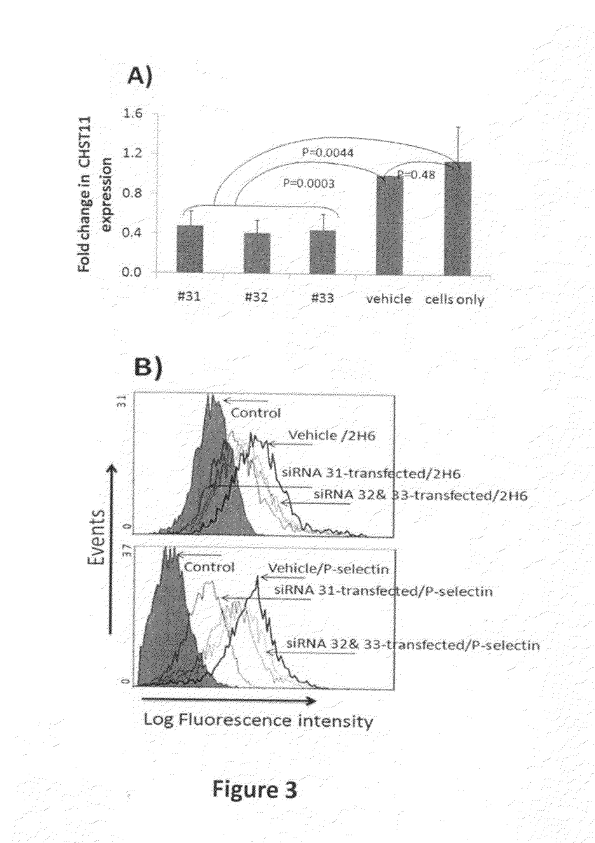

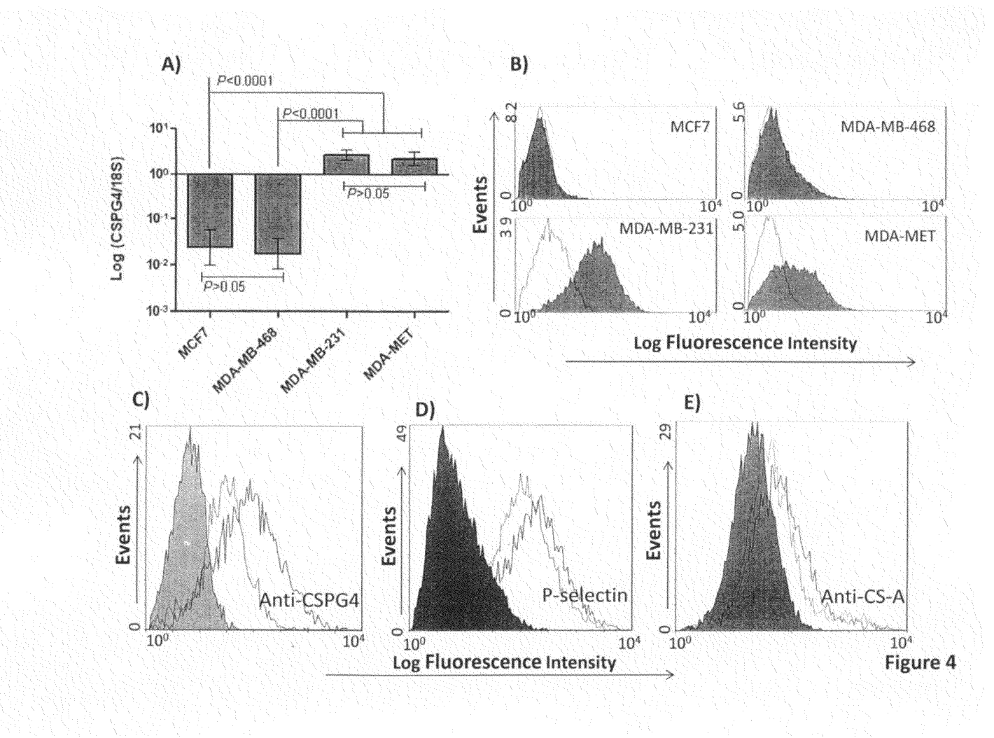

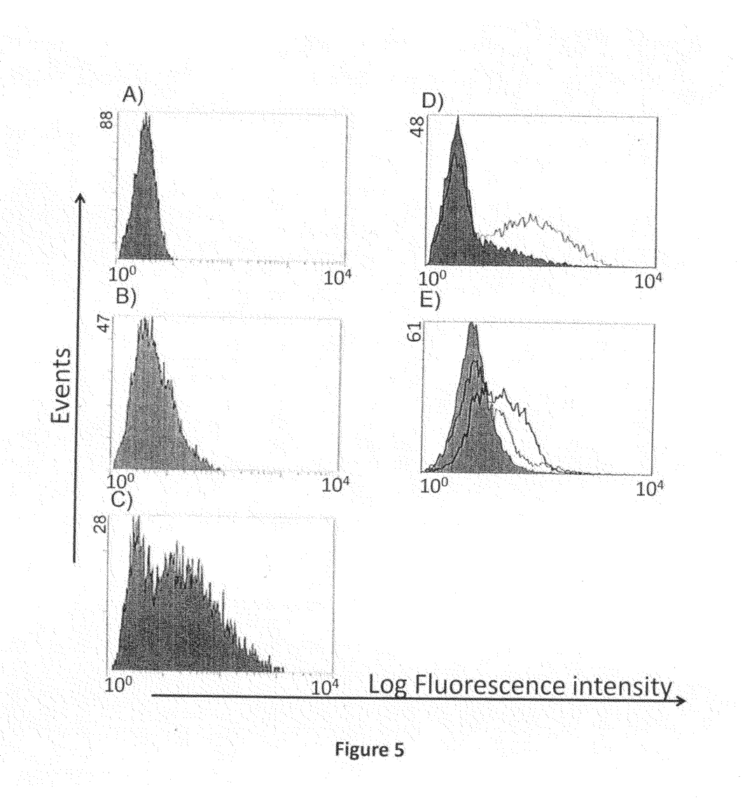

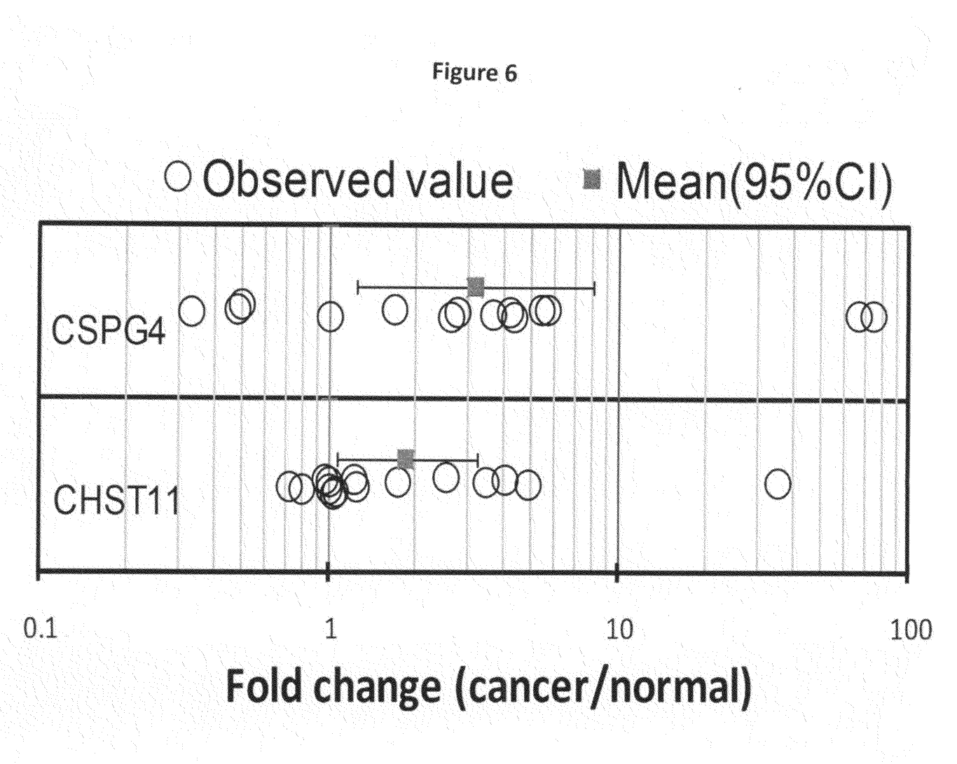

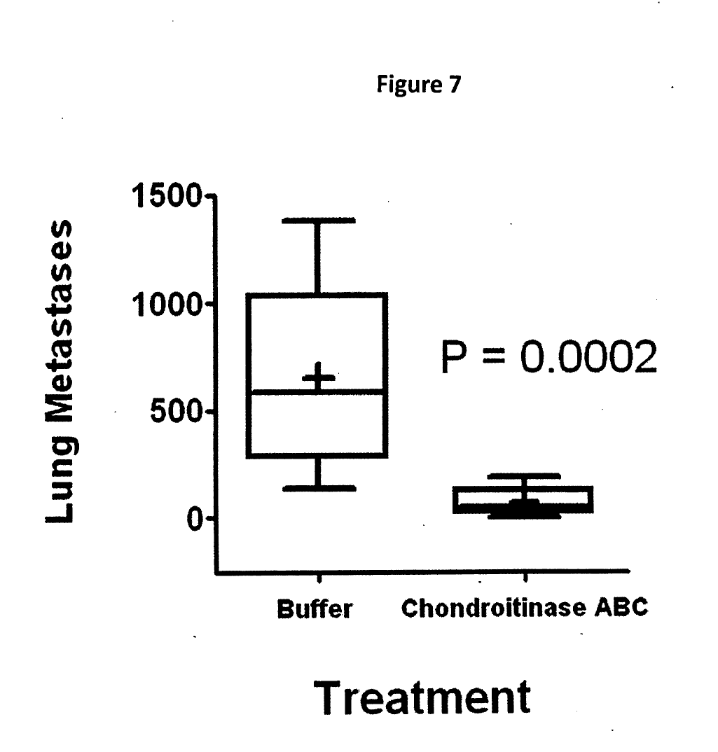

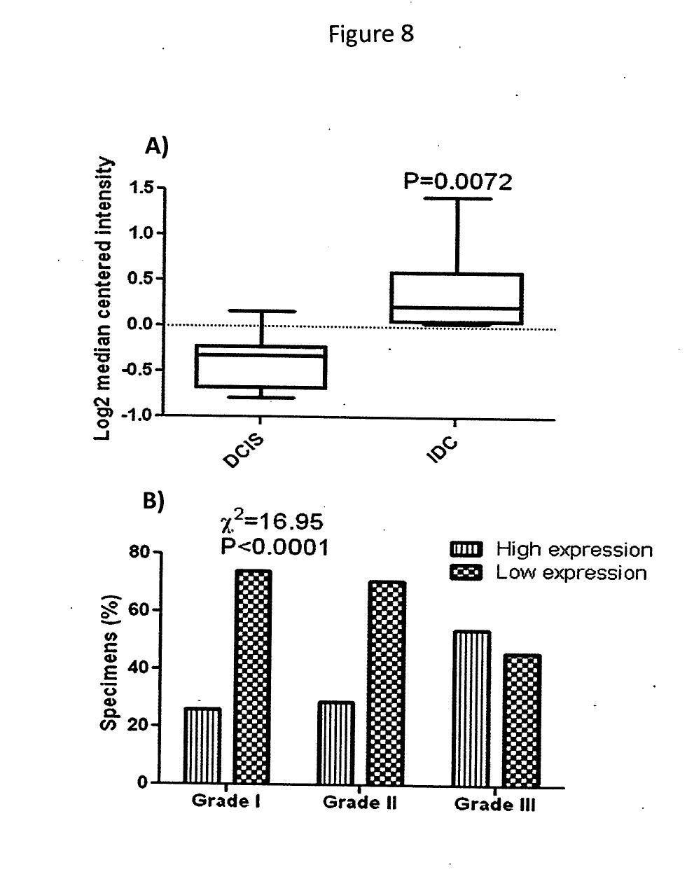

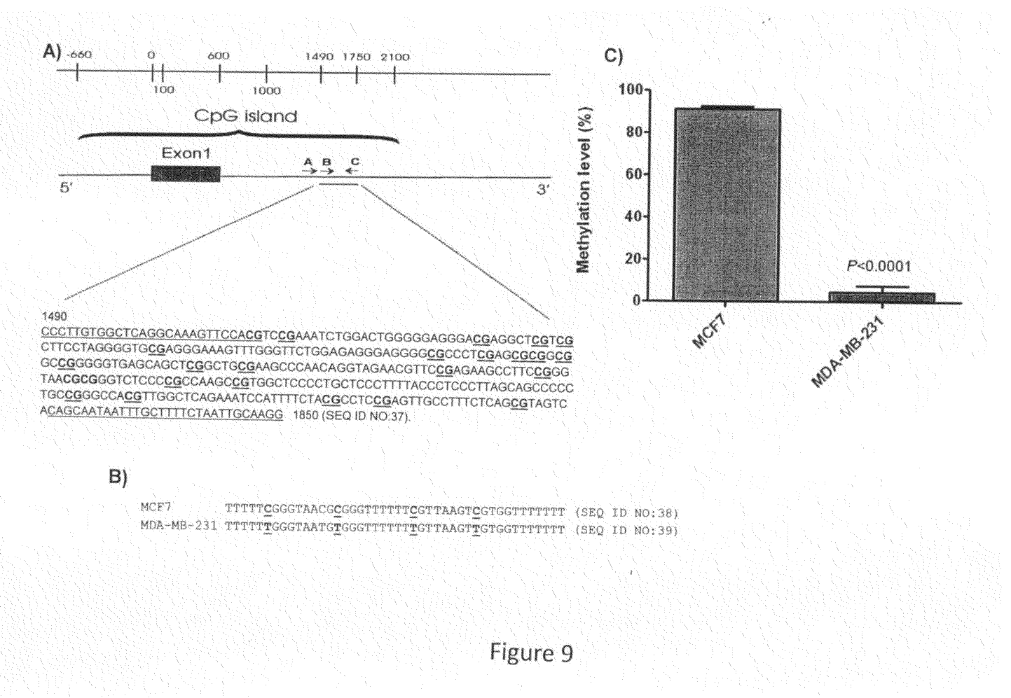

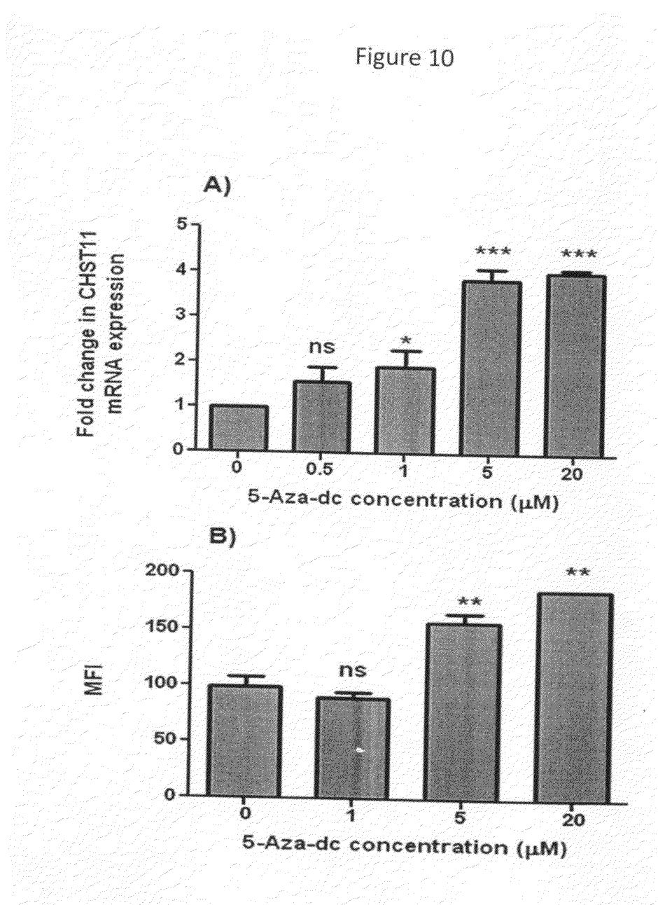

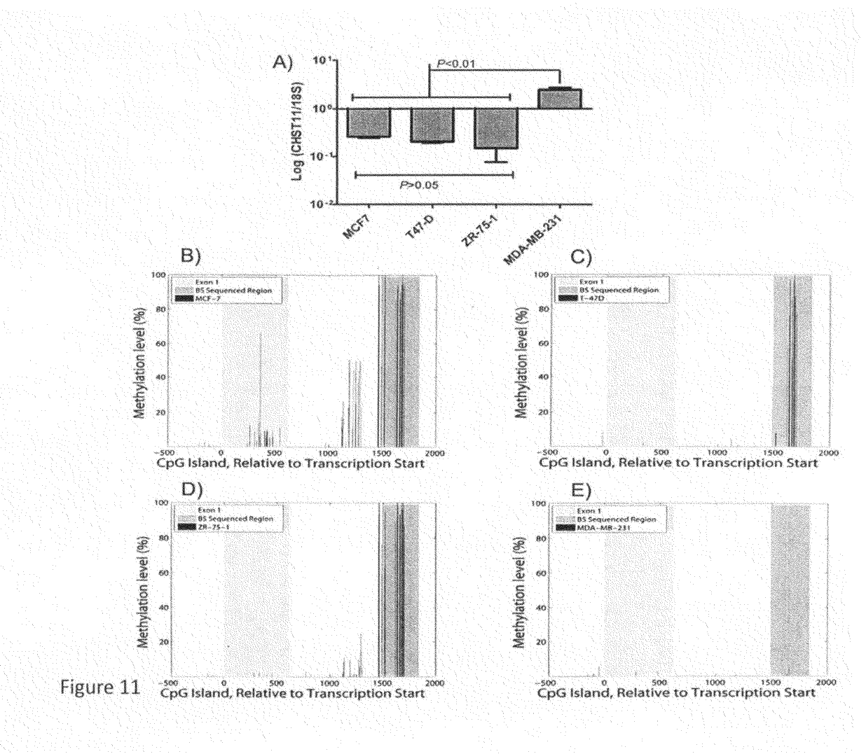

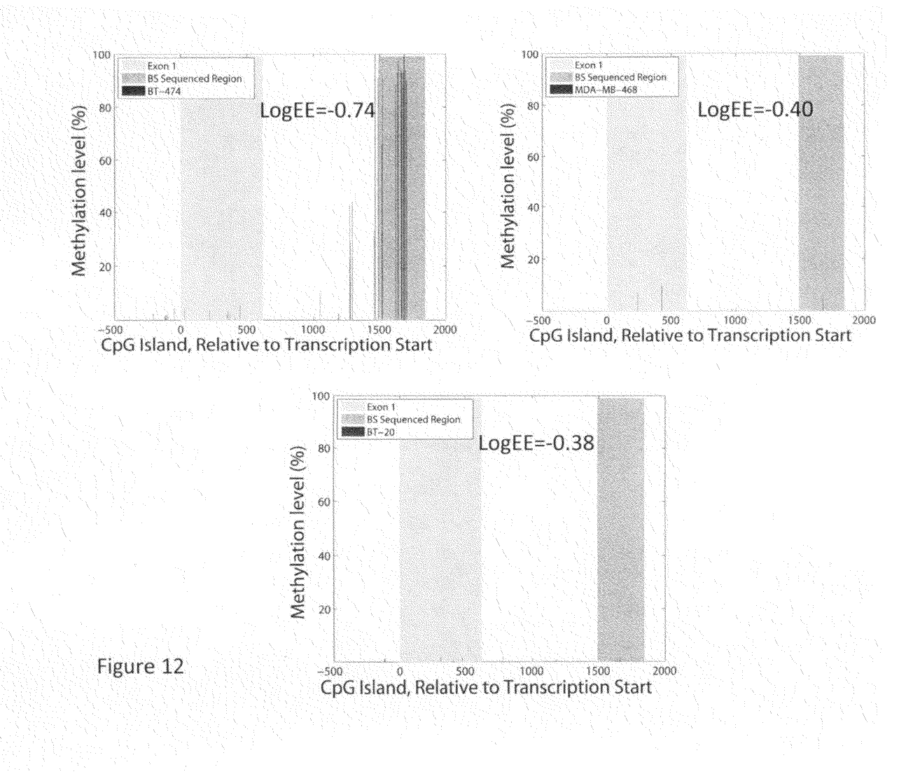

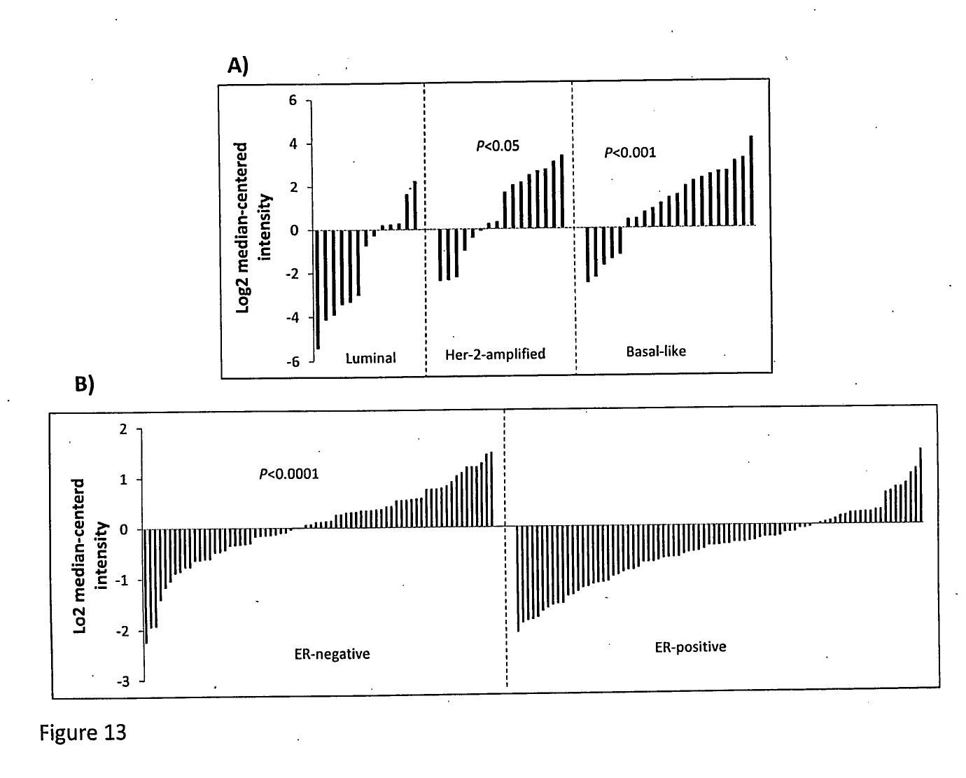

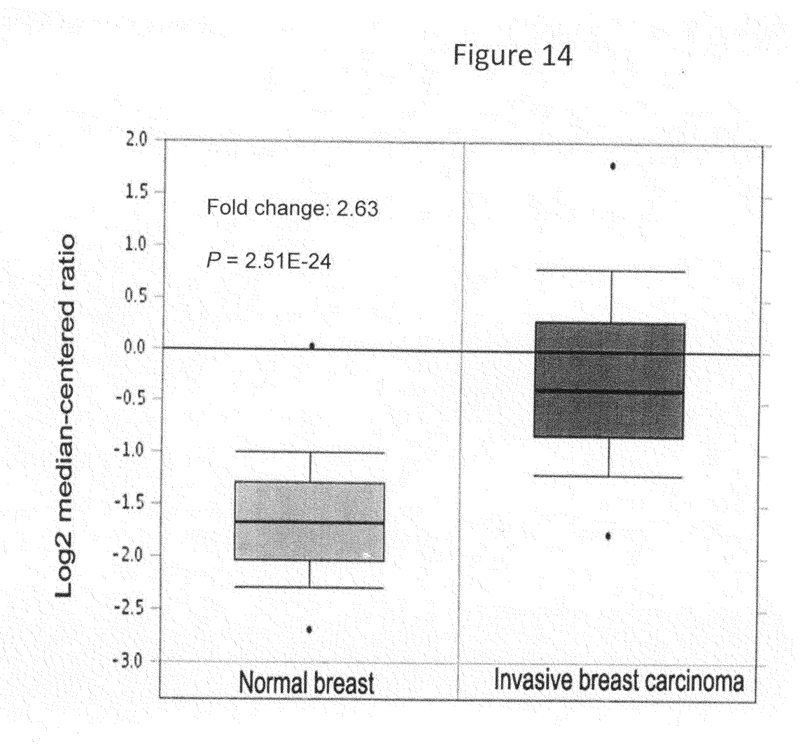

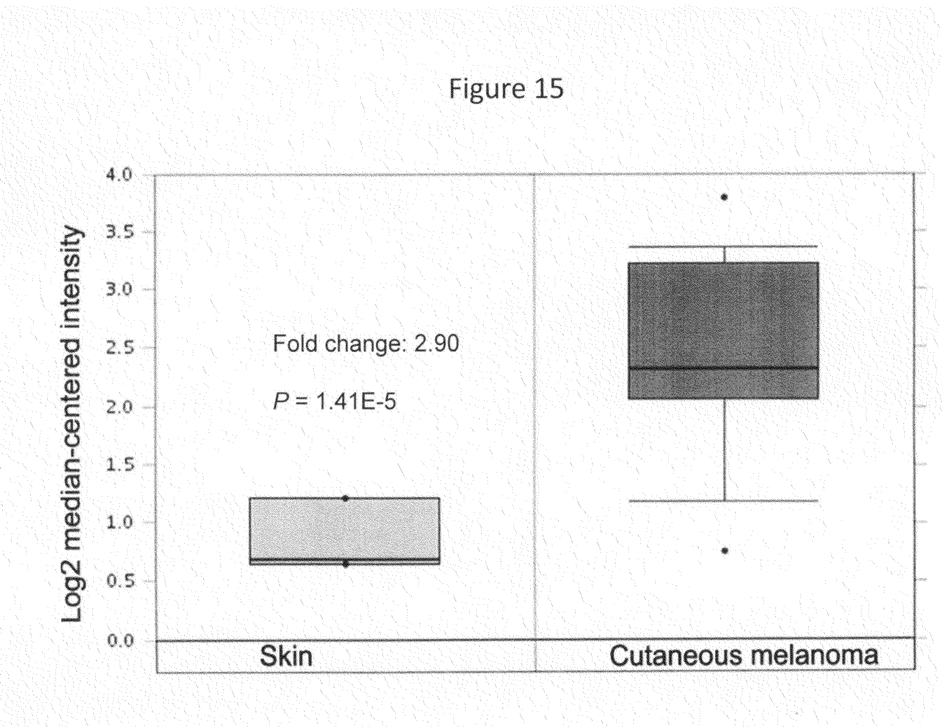

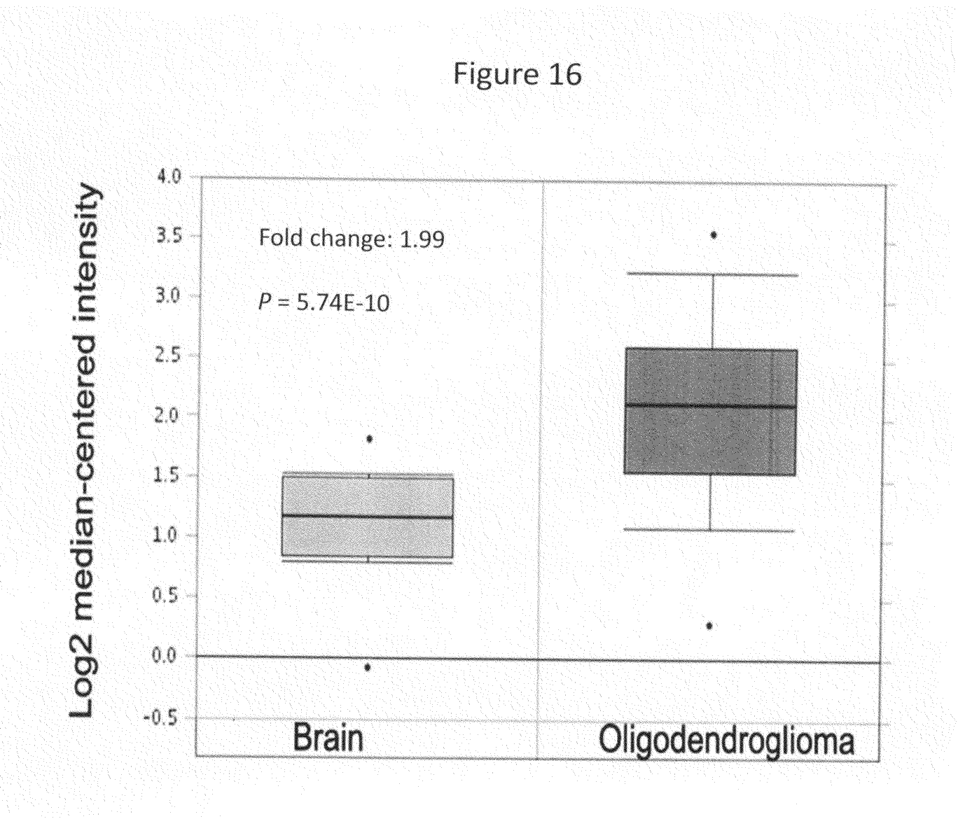

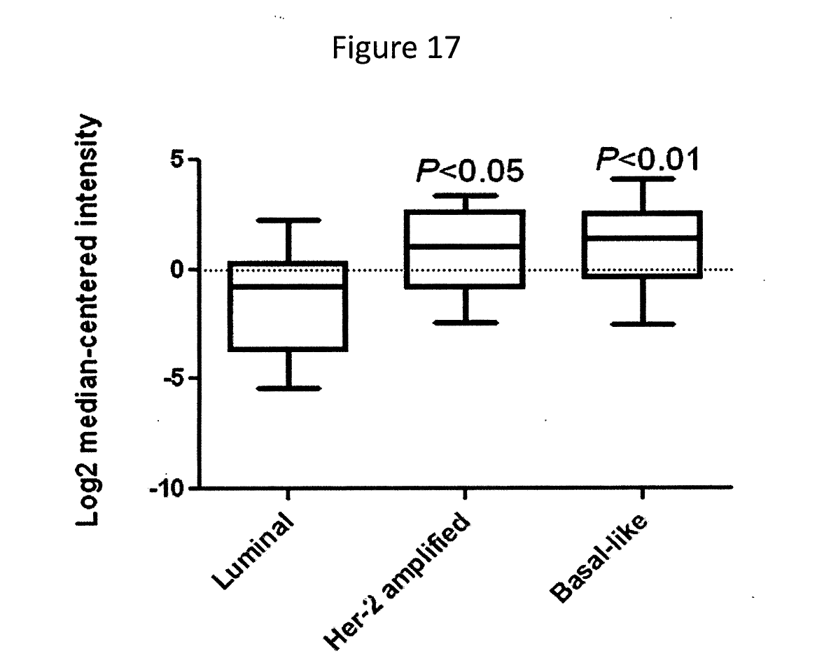

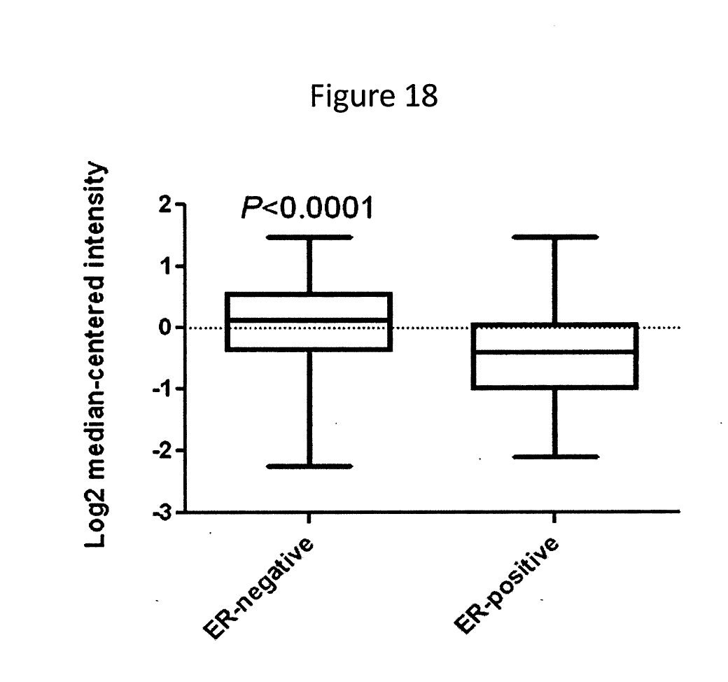

Our data indicate that CHST11 is required for P-selectin binding. To confirm a role of 4-O sulfated structures in P-selectin binding, the expression of CHST11 in MDA-MB-231 cells was inhibited by siRNA. We observed that CHST11 mRNA levels, anti-CS-A binding and P-selectin binding were all significantly reduced upon treatment with the three siRNAs tested (FIGS. 3A, B). Transfection with siRNA #31 (Table 1) showed the highest inhibitory effect on binding of anti-CS-A and recombinant P-selectin (FIG. 3B). In three independently conducted experiments, transfection with siRNA #31 significantly reduced the mean fluorescence intensity for anti-CS-A (P≦0.015) and P-selectin (P≦0.001) binding, as compared with vehicle-treated cells.

We repeated the CHST11 siRNA assay and did not observe any effect on the CSPG4 transcript or its surface expression as assayed by anti-CSPG4 mAb (Additional file 1). GAPDH siRNA was used as control in siRNA assays and reduced GAPDH mRNA by 75% in multiple assays (data not shown). Treatment of MDA-MB-231 cells with GAPDH siRNA did not inhibit CHST11 expression (data not shown). CHST11 siRNA sequences did not affect expression of GAPDH (data not shown). Therefore the expression of CS-A and binding of P-selectin to this cell line depends on the expression of the CHST11 gene.

### CSPG4 Expressed on Aggressive Breast Cancer Cells Functions as a P-Selectin Ligand Through its CS-GAGs

Because CS/DS-GAG expression and function depends on the composition of PGs expressed, studying the nature of the PG(s) involved in presentation of P-selectin-reactive CS/DS is important. Such studies should help us understand the functional consequences of CS/DS-GAG, PG and P-selectin interactions and provide data that may, in future studies, be used to manipulate the expression of the polysaccharide by targeting the core protein(s). Several membrane PGs, including SDC-1, SDC-4, NRP-1, and CSPG4 can potentially present GAG chains on the surface of tumor cells [53-55]. CSPG4 is the only cell surface PG that is exclusively decorated with CS-GAGs [41] and therefore, it may play a major role in forming cell surface CS-GAGs. We compared the expression of CSPG4 in the above described human breast cancer cell lines. The results of qRT-PCR indicate that the less aggressive epithelial-like cell lines MCF7 and MDA-MB-468 did not express CSPG4, while the gene was highly expressed in the highly aggressive mesenchymal-like cell lines MDA-MB-231 and MDA-MET (FIG. 4A). Flow cytometry analysis further confirmed that the expression of CSPG4 was high in aggressive cell lines MDA-MB-231 and MDA-MET with almost no expression detected in the less aggressive cell lines MCF7 and MDA-MB-468 (FIG. 4B).

Because CSPG4 is abundant on aggressive cells, we hypothesized that it may function as a major core protein presenting CS-A and CS-related P-selectin ligands. We used CSPG4 siRNA to inhibit CSPG4 expression (Additional file 2). Inhibiting CSPG4 transcript in turn inhibited cell surface expression of the PG (FIG. 4C), and that led to a drop in P-selectin (FIG. 4D) and anti-CS-A binding (FIG. 4E). We examined the transcript levels for CHST11 after transfection with CSPG4 siRNA (Additional file 2). Inhibition of expression of CSPG4 did not affect the expression of CHST11, indicating independence of expression of these two genes. This suggests that a decrease in CS-A after CSPG4 siRNA treatment (FIG. 4E) is due to reduced expression of CSPG4 and not of CHST11. These data suggest that CSPG4 participates in forming P-selectin ligands on the surface of highly aggressive human breast cancer cells, but obviously it is not the only PG involved in CS-GAG presentation. Others have reported the expression of syndecans and NRP-1 in these breast cancer cell lines and it is known that these PGs can present CS-GAGs [36-38, 54-56]. To confirm, we further examined the expression of SDC-1, SDC-4, NRP-1, and CSPG4 by qRT-PCR (Table 3). The expression of SDC-1 was lower in cells with highest metastatic potential and the expression of NRP-1, SDC-4 and CSPG4 was significantly higher in the most aggressive cells. The abundance of CS-A expression in MDA-MB-468 (a cell line with intermediate aggressiveness (FIG. 2A), combined with the lack of CSPG4 expression in this cell line (FIG. 4A, B), suggest that CS chains on this cell line are probably presented by other PGs (Table 3).

In order to confirm that P-selectin binds to CSPG4 we used CSPG4-transfected M14 melanoma cell line available in the lab. In prescreening of the M14 cell line, it appeared that this cell line expresses CHST11 with low binding of anti-CS-A 2H6 mAb and P-selectin, indicating that this cell line and its transfected version are excellent candidates for studying participation of CSPG4 and its CS GAGs in P-selectin binding. We observed that anti-CSPG4 225.28 mAb reacted with the transfected cells (M14-CSPG4) but not with mock-transfected M14 cells (FIG. 5A, B, C). Anti-CS-A 2H6 mAb reacted with M14-CSPG4 but not with mock-transfected M14 cells (FIG. 5D). P-selectin also reacted with M14-CSPG4 cells but not mock-transfected M14 cells, and treatment of M14-CSPG4 cells with chondroitinase ABC reduced the reactivity (FIG. 5E). Pre-incubation of cells with 225.28 did not inhibit P-selectin binding (data not shown). These data suggest that CSPG4, via its CS chain, serves as a P-selectin ligand on the cell surface and that anti-CSPG4 225.28 mAb does not react with the GAG part of CSPG4. The data further suggest that overexpression of both CSPG4 and CHST11 genes in tumor cells may contribute to a superior metastatic potential.

TABLE 3
Ratio of mRNA for some PG genes to 18S RNA

| | | MDA- | | |
|---|---|---|---|---|
| Antigen | MCF7 | MB-468 | MDA-MB-231 | MDA-MET |
| CSPG4 | 0.02 (±0.006) | 0.03 (±0.02) | 2.43 (±0.56) | 2.15 (±0.8) |
| NRP-1 | 0.96 (±0.10) | 1.00 (±0.08) | 1.55 (±0.20) | 1.96 (±0.07) |
| SDC-1* | 0.45 (±0.07) | 2.09 (±0.18) | 0.22 (±0.03) | 0.13 (±0.04) |
| SDC-4** | 0.58 (±0.08) | 0.86 (±0.07) | 1.50 (±0.30) | 1.90 (±0.11) |
| ER1 | 4.84 (±0.59) | 0.12 (±0.03) | 0.14 (±0.03) | 0.11 (±0.02) |

Gene expression was measured by qRT-PCR assays, repeated 3 times for each gene and each cell line and averages (±standard deviations) are shown. One-way ANOVA with Tukey's post-hoc procedure on log-transformed data was used for statistical analysis as described in Materials and Methods. Bold entries are statistically different (at P < 0.05) than non-bold entries in each row.
*The expression of SDC-1 in MDA-MB-468 cells is significantly higher than that in MDA-MB-231 and MDA-MET cells.
**The expression of SDC-4 in MDA-MB-468 cells is significantly less than that in MDA-MB-231 and MDA-MET cells. The expression of ER1 was used as a control knowing that MCF7 is ER-positive and the other three cell lines are ER-negative. CSPG4, Chondroitin Sulfate Proteoglycan 4;
ER1, Estrogen Receptor 1;
NRP-1: Neuropilin-1;
SDC-1, Syndecan-1;
SDC-4, Syndecan-4.

### CHST11 and CSPG4 are Overexpressed in Malignant Tissues of Breast Cancer Patients

In order to establish a translational relevance, we examined the expression of CSPG4 and CHST11 in specimens from breast cancer patients to compare the level of expression of these genes between normal and malignant tissues. Frozen sample-pair specimens from 15 breast cancer patients diagnosed with invasive ductal carcinoma were obtained from the UAMS tissue bank. In each sample pair, a tumor-containing sample was matched with tumor-free tissue from the same donor. We observed that these genes were overexpressed in tumor-containing tissues versus normal tissues (FIG. 6). CSPG4 and CHST11 expression showed an increase in tumor tissue over normal tissue in 10 out of 14 sample pairs and 8 out of 15 sample pairs, respectively. CSPG4 was elevated 3.2-fold (P<0.02) in tumor tissue over normal tissue among 14 subjects, while CHST11 was elevated 1.8-fold (P=0.034) in tumor over normal among 15 subjects (FIG. 6). Gene overexpression was detected in both ER-positive and ER-negative samples, but the majority of specimens with increased expression of CHST11 (seven out of eight), were HER2-neu-negative. These data suggest that despite the stromal expression of both CHST11 and CSPG4 genes the expression can be targeted specifically in aggressive tumors for therapeutic purposes. In this regard P-selectin binding to a relevant receptor on tumor cells may have profound impact on tumor cell dissemination and understanding the detail of such interaction may lead to development of novel anti-metastatic approaches.

### CS-GAG Removal Inhibits Tumor Metastasis

We have previously suggested a role for CS/DS-GAGs in metastasis of the murine mammary cell line 4T1 [24]. To directly link CS/DS-GAGs to tumor metastasis, we examined whether removal of the cell surface CS/DS-GAGs affects metastasis of 4T1 cells in vivo. Here we demonstrate that removing CS/DS-GAGs by treating cells with chondroitinase ABC attenuated lung metastases in the 4T1 murine tumor model (FIG. 7). Chondroitinase treatment of cells prior to tail-vein injection significantly reduced lung metastases (P=0.0002). The data indicate a significant role for tumor surface CS/DS-GAGs in establishing lung metastases in this breast cancer model and further support a likely role for P-selectin interaction with CS/DS-GAG in breast cancer metastasis.

### Discussion

Tumor cell dissemination by platelets leads to colonization of cancer cells to secondary organs resulting in poor prognosis and high mortality of cancer patients. P-selectin is present on activated platelets and endothelial cells while CS/DS-GAGs on the surface of breast cancer cells with high metastatic potential serve as P-selectin ligands [24]. The role of P-selectin in heterotypic adhesion is a critical component determining the efficiency of tumor cell dissemination [16, 17]. The study of P-selectin-reactive molecules on tumor cells is crucial for the assessment of metastatic risk and the development of possible ways of dealing with metastatic disease. Such studies are needed to ultimately reveal the functional consequences of P-selectin/ligand interaction in tumor progression, making specific links between platelets and tumor metastasis.

Our results suggest the CHST11 gene as a major player in production of such P-selectin ligands. The results using CHST11 siRNA further suggest that among the chondroitin sulfotransferases tested, CHST11 expression has a rate limiting role in constructing both CS-A chains and P-selectin ligands on MDA-MB-231 cells. This data directly links expression of the CHST11 gene to P-selectin-reactive GAGs on this cell line and has significant implications for further functional studies. CS-A is an immediate product of CHST11 expression and is considered a precursor in forming CS-E units [57]. P-selectin has been shown to bind to CS-E [23]. We have also shown that exogenous CS-E inhibits P-selectin binding to cancer cells [24]. While we do not rule out participation of CS-E in P-selectin binding, our data do not support CS-E as the P-selectin reactive GAG unit on these cells. The combined high expression of these two genes may associate with P-selectin-reactive glycans and aggressiveness. This possibility should be investigated in future studies. However, the data indicate that the expression of the CHST11 gene in tumor cells is associated with synthesis of P-selectin ligands and a metastatic phenotype. Others have suggested a role for CS-A in tumor progression and metastasis in a melanoma model [46]. However, our data suggest that surface presentation of CS-A may be required but is not sufficient for a metastatic phenotype to occur. Besides its role in constructing CS-A, CHST11 also plays a role in chain elongation and production of more CS [57]. Chain elongation activity of CHST11 with a fine balance in the expression of other enzymes might be needed for constructing conformational epitopes. Thus, CHST11 activity may lead to larger CS polymers with multiple sequences embedded with distinct sulfation patterns resulted from activity of multiple sulfotransferases, leading to the production of conformational epitopes or highly concentrated sequences with specific reactivities. Thereby, the expression of CHST11 may correlate better with the tumor cells' aggressive phenotype than does the prevalence of any particular CS isomers.

Interestingly, our current results implicate CSPG4 in the presentation of CS moieties as P-selectin ligands. CSPG4 exclusively presents CS-GAGs, and the data described here suggest that these structures interact with P-selectin and, therefore, may contribute to distant metastasis of tumor cells. Our data indicate that P-selectin binds to CSPG4 through CS-GAGs and that CSPG4 is involved in P-selectin binding to CSPG4-expressing breast cancer cells. However, other PGs may also participate in P-selectin binding as expression of SDC-4 and NRP-1 is also higher in MDA-MB-231 and MDA-MET. Lack of expression of CSPG4 in MDA-MB-468 further suggests that anti-CS-A and P-selectin binding to these cells is probably due to expression of other PGs and not CSPG4. However, because of the role of CSPG4 in signaling and tumor phenotype, we speculate that its interaction with P-selectin may lead to an exclusive tumor cell activation and consequently survival in circulation. Therefore, concerted upregulation of CSPG4 and CHST11 may induce expression of a unique molecular entity that may increase the metastatic capabilities of tumor cells. More studies are needed to understand the consequences of P-selectin binding to CS-GAGs of multiple PGs and to reveal how and at what stage of the metastatic cascade the CS-GAGs and their carrier proteins contribute to metastasis.

We have shown glycan interactions with P-selectin and the significance of P-selectin binding in metastasis of a murine mammary cell line [24, 52]. Our previous findings support the concept that CS chains promote survival in the circulation and tumor cell extravasation via P-selectin-mediated binding to platelets and endothelial cells. In the current study, the significance of the cell surface expression of CS-GAGs in a breast cancer model is established. The data demonstrate that enzymatic removal of the CS chains significantly attenuated formation of lung metastases in a highly metastatic mammary cell line. Others have shown that P-selectin ligands are critical components of heterotypic adhesion, determining the efficiency of tumor cell dissemination [16, 17]. Sugahara's group demonstrated that highly sulfated CS-GAGs, in particular CS-E, are involved in metastasis of murine lung carcinoma and osteosarcoma cells [58, 59]. However, our data suggest a role for 4-0 sulfation of chondroitin in the metastatic phenotype. CHST11 expression results in the synthesis of a particular Chonroitin sulfate important for cell-cell adhesion and metastasis. Moreover, the data suggest that the presence of CS/DS-GAGs may not be sufficient for a phenotype with high metastatic capacity to occur. The data emphasize a combination of the polysaccharide and a core protein as a pro-metastatic entity or prometastatic code. Future studies are needed to understand the contribution of each PG in P-selectin mediated tumor cell behavior.

We further showed that the expression of CHST11 and CSPG4 is elevated in tumor tissues from breast cancer patients. Consistent with our data, CHST11 expression has been shown to be greater in human breast carcinoma compared to normal breast tissue [60] and in malignant plasma cells from myeloma patients compared to normal bone-marrow plasma cells [61].

The current research should lead to future studies of functional relationships between CS and tumor progression. Existing knowledge and further mechanistic studies might suggest CS-GAGs and their presenting PGs as targets for antimetastatic therapies. In support of work done in melanoma [62] and recent studies in breast cancer [39], the studies outlined here strongly suggest that CSPG4 can be an available target for immunotherapy of breast cancer. However, in order to efficiently block tumor cell dissemination by interrupting P-selectin/CS interaction, targeting any single PG does not seem enough as other PGs can probably compensate and support metastatic processes. In this regard, global targeting of specific CS isomers may be a particularly effective approach.

Breast cancer cell surface is decorated with CS-GAGs and due to tumor-specific expression patterns of chondroitin sulfotransferases and PGs, the composition and binding specificity of these polysaccharides differ from those of normal tissues. Therefore, these molecules and their interaction with P-selectin should be considered as viable targets for the development of novel therapeutic strategies.

### Conclusions

This study demonstrates the significance of CS-GAGs in the lung colonization of an aggressive murine mammary cell line. The study reveals that CSPG4 can serve as a P-selectin ligand through its CS chain and that the expression of the CHST11 gene controls P-selectin reactive CS-GAGs formation. The data suggest that CS-GAGs, their biosynthetic pathway, or the core protein carrying them can be potential-targets for the development of therapeutic strategies for treatment of aggressive breast tumors. The knowledge and perspective gained from this line of research together with further mechanistic studies may pave the road to target CS-GAGs, their carrier PGs and their interaction with P-selectin as novel antimetastatic therapies.

### Example 2

### The Expression and Methylation of the Carbohydrate (Chondroitin 4) Sulfotransferase-11 Gene in Breast Cancer Cells

### Summary

We have shown that the expression levels of carbohydrate (chondroitin 4) sulfotransferase-11 (CHST11) correlate with the expression of P-selectin-reactive surface chondroitin sulfate (CS) and aggressiveness of human breast cancer cells. This study was performed to further evaluate the expression of the CHST11 gene in breast cancer and determine whether the expression of this gene is controlled by DNA methylation. Expression analysis of Oncomine datasets revealed that the expression of CHST11 is significantly higher in cancer tissues with the expression levels correlating with tumor progression and aggressiveness. Our data demonstrate that the expression of CHST11 and its immediate product chondroitin sulfate A (CS-A) was high in the estrogen receptor (ER)-negative MDA-MB-231 cell line and very low in ER-positive MCF7 cells. We observed very low levels of DNA methylation in a CpG island of CHST11 in ER-negative cells but very high levels in the same region in ER-positive cells. Treatment of MCF7 cells with 5AzadC increased the expression of CHST11 and CS-A in a dose-dependent manner. The data suggest that breast cancer cells may use DNA methylation as a mechanism to control the expression of CHST11. The results demonstrate the utility of CHST11 expression and its DNA methylation status as potential biomarkers in breast cancer.

### Introduction

CHST11 is a key enzyme in the biosynthesis of chondroitin sulfates (CS) and its action on chondroitin chains can lead to the production of chondroitin sulfate A, B and E (CS-A, CS-B, CS-E) (Mikami et al. 2003; Uyama et al. 2006). Several studies, including ours, suggest roles for CS-A, CS-B, and CS-E in tumor progression and metastasis (Iida et al. 2007; Monzavi-Karbassi et al. 2007; Li et al. 2008). Recently, we reported that the expression levels of the CHST11 gene correlate with CS-A expression, P-selectin binding and aggressiveness of human breast cancer cell lines (Cooney et al. 2011). We have shown that removing CS chains and blocking P-selectin interaction with breast cancer cells significantly attenuates metastasis (Monzavi-Karbassi, Stanley et al. 2007; Cooney, Jousheghany et al. 2011). Based upon our results we hypothesize that CHST11 expression, by affecting the cell surface CS profile and construction of P-selectin-reactive epitopes, plays a significant role in the metastatic behavior of breast cancer. Therefore, for a potential future use in the development of therapeutic or diagnostic strategies targeting CHST11 gene, understanding the mechanisms controlling CHST11 expression is important.

Aberrant DNA methylation is a mechanism controlling gene expression and is involved in tumor initiation and progression (Feinberg et al. 1988; Narayan et al. 1998; Baylin et al. 2001; Ehrlich 2002; Szyf et al. 2004; Cooney 2008). The expression of several glycosaminoglycan (GAGs) sulfotransferases is associated with DNA methylation (Miyamoto et al. 2003; Bui et al. 2010; Tokuyama et al. 2010). A clear association between DNA methylation, expression levels of CHST11 and its final products such as CS-A and metastatic potential has not been established.

We show here and in Example 1 that the expression levels of CHST11 can predict progression of the breast disease and more aggressive phenotypes. Our investigation of the methylation status of CHST11 in breast cancer cells clearly demonstrates an association between lack of expression of this gene and hypermethylation of a CpG island of its DNA. The 5AzadC treatment of less aggressive epithelial-like luminal human MCF7 cells lacking CHST11 expression, led to a dose response increase in the expression of the gene and its product CS-A. Given the role of CS in cell-cell interaction resulting in tumor growth and metastasis, these findings have significant implications for the development of novel prognostic and therapeutic approaches targeting CS.

### Materials and Methods

Reagents.

Anti-CS-A mAb 2H6 was from Associates of Cape Cod/Seikagaku America (Falmouth, Mass.). Fluorescence-conjugated, anti-mouse IgM and 5-aza-2′-deoxycytidine (5AzadC) were from Sigma (St. Louis, Mo.). Primers were from Integrated DNA Technologies (IDT, Coralville, Iowa). Real-time PCR reagents were from Applied Biosystems (Foster City, Calif.). TRIzol reagent was from Invitrogen (Carlsbad, Calif.), FailSafe PCR PreMix Selection kits and Enzyme Mix were from Epicentre Biotechnologies (Madison, Wis.). Alkaline phosphatase (rAPid) was from Roche (Nutley, N.J.).

Analysis of Oncomine Cancer Gene Microarray Database.

For comparison of CHST11 mRNA expression between cancer and normal tissues, between ductal carcinoma in situ (DCIS) and invasive ductal carcinoma (IDC), and between tumors of different nuclear grades, we selected datasets from the Oncomine cancer microarray database (Compendia Biosciences; Ann Arbor, Mich., USA). This database was also used to compare expression levels of CHST11 between ER-negative and ER-positive patients' specimens and among a panel of cell lines that represent luminal, Her-2-amplified and basal-like molecular subtypes of breast cancer. Log-transformed, median-centered and normalized expression values (Rhodes et al. 2004) were extracted, analyzed and graphed accordingly.

Cell Lines.

Cell lines MCF7, MDA-MB-231, MDA-MB-468, T47-D, and ZR-75-1 were from ATCC (Manassas, Va.). MDA-MB-231, MDA-MB-468, T47-D, and ZR-75-1 cells were cultured in DMEM supplemented with 10% heat-inactivated fetal bovine serum (Life Technologies), 50 units/mL penicillin, and 50 μg/mL streptomycin. MCF7 were grown as described before (Cooney, Jousheghany et al. 2011). Cells are checked every six months to be free from Mycoplasma contamination using the MycoAlert® Mycoplasma Detection Kit (Lonza Rockland Inc., Rockland, Me.).

Total RNA Isolation and Quantitative Real Time RT-PCR (qRT-PCR).

Total RNA was isolated from cultured cells using TRIzol reagent, following the manufacturer's instructions. The quantity and quality of the isolated RNA was determined, using an Agilent 2100 Bioanalyzer (Palo Alto, Calif.). One μg of total RNA was reverse-transcribed using random-hexamer primers with TaqMan Reverse Transcription Reagents (Applied Biosystems). Reverse-transcribed RNA was amplified with SYBR Green PCR Master Mix (Applied Biosystems) plus 0.3 μM of gene-specific upstream and downstream primers during 40 cycles on an Applied Biosystems 7900 HT Fast Real Time System. Data were analyzed by absolute and relative quantification. In absolute quantification, data were expressed in relation to 18S RNA, where the standard curves were generated using pooled RNA from the samples assayed. In relative quantification, the 2^{(-Delta Delta C(T)}) method was used to assess the target transcript in a treatment group to that of untreated control using expression of an internal control (reference gene) to normalize data (Livak and Schmittgen 2001). Expression of GAPDH was used as internal control. The primer sequences are shown in Table 4.

Digital Gene Expression.

Second generation mRNA-sequencing data generated with the 75 by directional protocol for the Illumina Idea Challenge (illumine.com/landing/idea) project was used to evaluate digital gene expression of CHST11. We followed the method of Mortazavi et al. (Mortazavi et al. 2008) to estimate the number of expressed transcripts of the CHST11 gene C_{CHST11} by taking into account the number of reads mapping to the gene N_{CHST11}, mapped gene length L_{CHST11}, total number of reads mapped on the known exons of the sequenced transcriptome N_{T}, and the total length of the mapped transcriptome L_{T} as:

$${C}_{CHST 11} = \frac{{N}_{CHST 11}}{{L}_{CHST 11}} \times \frac{{L}_{T}}{{N}_{T}} .$$

We used this equation to estimate the number of expressed copies of the regions analyzed by the RT-PCR.

5AzadC Treatment.

5AzadC was dissolved in ice-cold phosphate-buffered saline, filter sterilized at 4° C. and the resulting solution used to treat the MCF7 cell line. For dose-response experiments, cells were harvested 5 days after the initial treatment. Cell growth medium was refreshed every other day.

Extraction and Bisulfite Modification of DNA.

DNA from cells was extracted as described before (Leakey et al. 2008). DNA was bisulfite modified with an Epitect Kit (Qiagen, Valencia, Calif.) using 300 ng of DNA per reaction. PCR was performed using a FailSafe PCR PreMix Selection kit and FailSafe Enzyme Mix. Each 25 μl PCR reaction included 1.0 μM of each primer, 2.5 units of the FailSafe Enzyme Mix and 12.5 μl of the FailSafe PCR Premixes A or C. Bisulfite-modified genomic DNA was amplified by seminested PCR using two sets of primers for part of intron1 that is within a CpG island spanning exon 1 of the CHST11 gene (Genbank NM_{—}000012 and exon 1 located with NM_{—}018413). The same amplification profile was used for both reactions of the seminested PCR: 1 cycle at 80° C. for 1 min, 1 cycle at 94° C. for 1 min; 1 cycle at (95° C. for 1 min, 54° C. for 1 min, 72° C. for 1 min); 1 cycle at (95° C. for 1 min, 53° C. for 1 min, 72° C. for 1 min); 1 cycle at (95° C. for 1 min, 52° C. for 1 min, 72° C. for 1 min); 1 cycle at (95° C. for 1 min, 51° C. for 1 min, 72° C. for 1 min); 36 cycles at (95° C. for 1 min, 50° C. for 1 min, 72° C. for 1 min); 72° C. for 5 min and cooling to 4° C.

A forward, outside primer and reverse primer were used for CHST11 for the first reaction (Table 4). A second, semi-nested, PCR was then performed on 1 μl of the amplificate (in a 25 μl PCR reaction) using a forward nested primer and the reverse primer from the first reaction (346 bp PCR product, Table 4). The primers were designed using MethPrimer web software (Li and Dahiya 2002, available from Urogene). The CpG island was defined using CpG Island Searcher set on the default criteria for defining CpG islands (Takai and Jones 2003) (cpgislands.us.edu/cpg.aspx).

TABLE 4
Primers used in this study

| Primer | Sequence |
|---|---|
| 18S forward | 5′ TTCGAACGTCTGCCCTATCAA 3′ (SEQ ID NO: 1) |
| 18S reverse | 5′ ATGGTAGGCACGGCGACTA 3′ (SEQ ID NO: 2) |
| CHST11 forward | 5′ TCCCTTTGGTGTGGACATCT 3′ (SEQ ID NO: 9) |
| CHST11 reverse | 5′ CACGTGTCTGTCACCTGGTC 3′ (SEQ ID NO: 10) |
| GAPDH forward | 5′ACAGTCAGCCGCATCTTCTT3′ (SEQ ID NO: 23) |
| GAPDH reverse | 5′ACGACCAAATCCGTTGACTC3′ (SEQ ID NO: 24) |
| CHST11 bisulfite | 5′ TTTGATTATTGTAGTTTTGGAGGAAAT 3′ (SEQ ID NO: 34) |
| outside, forward | |
| CHST11 bisulfite | 5′-CCTTACAATTAAAAAAACAAATTATTACTA-3′ (SEQ ID NO: 35) |
| reverse | |
| CHST11 bisulfite | 5′-TTTTTGTGGTTTAGGTAAAGTTTTA-3′ (SEQ ID NO: 36) |
| nested, forward | |

18S, 18S ribosomal RNA;
CHST11, Carbohydrate (Chondroitin 4) Sulfotransferase 11;
GAPDH, Glyceraldehyde-3-phosphate dehydrogenase.

Bisulfite genomic sequencing (BGS) and methylation level quantification.

BGS was performed as described before (Leakey, Zielinski et al. 2008) with the following minor modifications. We used rAPid alkaline phosphatase and PCR products were sequenced using the nested forward (upstream) CHST11 primer. We also analyzed DNA methylation in the region spanning more than 1.5 kb of the CHST11 CpG island with reduced representation bisulfite sequencing (RRBS, (Meissner et al. 2008; Smith et al. 2009)) data generated on breast cancer cell lines for the Illumina Idea Challenge (illumina.com/landing/idea). RRBS selects DNA fragments up to 220 by in the vicinity of MspI recognition sites (C.CGG) (Meissner, Mikkelsen et al. 2008). We only considered CpG dinucleotides with coverage larger than 10×. The percentage of methylation was inferred from the number of methylated reads divided by the sum of methylated and unmethylated reads. All reads were mapped to the hg18 release of the human genome Second generation sequencing DNA methylation results were plotted in Matlab (available from Mathworks).

Statistical Analysis.

The Chi-square test for trend was performed for gene expression across tumor grade. The Mann-Whitney test was performed to compare gene expression between DCIS and IDC and between ER-negative and ER-positive cancer specimens. For 5AzadC induced fold change in gene expression, the mRNA levels of the non-zero doses for each transcript and experimental replication were normalized to that of the zero-dose control, transformed to their base-10 logarithms, and analyzed for trend with dose via one-way ANOVA. For comparison of gene expression between cell lines, the raw amount for each mRNA was log transformed and normalized to the control mRNA (18S) amount, and analyzed via one-way ANOVA with Tukey's post-hoc procedure. The same was used to compare gene expression between subtypes of cell lines. Percent methylation was Arcsin transformed and then analyzed via Mann-Whitney test. Statistical analyses were performed using Excel (Microsoft, Seattle, Wash.) or GraphPad Prism version 5.00 for Windows, (GraphPad Software, San Diego, Calif. All P values were 2-sided.

### Results

### CHST11 is Overexpressed in Breast Cancer Tissues and the Expression Levels Associate With Progression and Tumor Grade.

We reported previously that CHST11 is overexpressed in tumor tissues versus normal breast tissues (Cooney, Jousheghany et al. 2011). Here, we confirmed the overexpression of CHST11 in malignant tissues, analyzing multiple datasets using the Oncomine database (Table 5). To investigate the association of CHST11 overexpression with tumor progression, we interrogated two separate datasets in the Oncomine database. In a dataset comparing DCIS with IDC (Schuetz et al. 2006), we found a significant increase in the expression of CHST11 in IDC samples (FIG. 8A). In another dataset examined (Gluck et al. 2011), the expression levels of CHST11 showed significant association with tumor grade (FIG. 8B), as the percentage of specimens with high expression of CHST11 increased with nuclear grade.

TABLE 5
CHST11 differential transcript expression in human
breast carcinomas extracted from multiple studies in Oncomine
microarray database

| | Comparison (specimen | Average fold | |
|---|---|---|---|
| Study+ | number in each group) | increase | P value |
| TCGA⁺⁺ | Invasive breast carcinoma | 2.6 | 2.51E−24 |
| | (76) Vs normal(61) | | |
| Finak | Invasive breast carcinoma | 3.6 | 9.43E−20 |
| | (53) Vs normal(6) | | |
| Richardson | Ductal breast carcinoma (40) | 2.2 | 2.00E−4 |
| | vs normal (7) | | |
| Gluck | Invasive breast carcinoma | 2 | 0.016 |
| | (154) vs. normal (4) | | |

+studies are addressed by author's last name.
⁺⁺TCGA, The Cancer Genome Atlas - Invasive Breast Carcinoma Gene Expression Data.

### A CpG Island in the CHST11 Gene Sequence is Hypermethylated in MCF7 and Hypomethylated in MDA-MB-231 Cells.

CS-A, the immediate product of CHST11 expression, has been found to play a role in metastasis and progression of cancer cells (Iida, Wilhelmson et al. 2007; Cooney, Jousheghany et al. 2011). We demonstrated previously that the level of expression of CHST11 and CS-A was high in highly metastatic MDA-MB-231 cells, while it was significantly lower in MCF7 cells as assayed by qRT-PCR and flow cytometry (Cooney, Jousheghany et al. 2011). To examine whether DNA methylation controls variation and regulation of CHST11 in breast cancer cell lines, we examined DNA methylation of the CHST11 sequence in a section of its CpG island covering part of its promoter, its first exon, and a portion of its first intron (FIG. 9A). Using BGS and the Mquant algorithm (Leakey, Zielinski et al. 2008) we determined methylation levels of ten CpG sites for MCF-7 and MDA-MB-231 cells (FIG. 9B, C). This revealed very high methylation levels (91%) averaged over 10 CpGs in the CHST11 sequence in MCF7 cells, and very low levels (5%) over the same 10 CpGs in MDA-MB-231 cells (FIG. 9C). These observations suggest that low expression of CHST11 in MCF7 cells is probably due to DNA hypermethylation. (Note that the sequence shown in FIG. 9B is a bisulfite treated, so all non-methylated Cs are converted to Ts. The sequences in FIG. 9B arise from the 1490-1850 sequence of FIG. 9A, but many of the Ts in panel B are Cs in panel A.)

Treatment of MCF7 cells with 5AzadC increases the expression of CHST11 and CS-A.

To further demonstrate that low expression of CHST11 in the MCF7 cell line is due to hypermethylation status of the CHST11 CpG island, the MCF7 cells were treated with 5AzadC and gene expression was evaluated. Upon 5AzadC treatment, we observed a significant increase in the expression of CHST11 mRNA (FIG. 10A) that paralleled surface expression of CS-A as assayed by flow cytometry using anti-CS-A 2H6 mAb (FIG. 10B). Together, the data suggest that low expression of CHST11 in the MCF7 cells is due to promoter hypermethylation, and that DNA hypomethylation in the more aggressive mesenchymal-like MDA-MB-231 cell line is permissive for overt CHST11 expression

### The CHST11 CpG Island is Hypermethylated in Other ER-Positive Breast Cancer Cells.

Our previous data suggest that CHST11 expression can be low in ER-positive and high in ER-negative breast cancer cell lines (Cooney, Jousheghany et al. 2011). To further explore this possibility we examined the expression of CHST11 in two other ER-positive cell lines, T47-D and ZR-75-1 by qRT-PCR (FIG. 11A) and flow cytometry (Table 6) and compared it with the expression levels of the gene in MCF7 and MDA-MB-231. We observed that the expression of CHST11 gene and CS-A in both of these cell lines were significantly lower than MDA-MB-231 and comparable with MCF7 cells.

TABLE 6
Summary of CHST11 gene expression and methylation status in human breast
cancer cell lines tested.

| | | | | CS-A | |
|---|---|---|---|---|---|
| | | | | expression(2H6 | |
| | | Molecular | CHST11 (qRT- | mAb binding by | DNA methylation |
| Cell line | ER status | subtype | PCR) | flow cytometry) | status |
| MCF7 | Positive | Luminal | Low | Low* | Hypermethylated |
| T47-D | Positive | Luminal | Low | Low++ | Hypermethylated |
| ZR-75-1 | Positive | Luminal | Low | Low++ | Hypermethylated |
| BT-474 | Positive | Her-2 | Low+ | Nd | Hypermethylated |
| | | amplified | | | |
| MDA-MB-468 | Negative | Basal-like | Intermediate | Intermediate to | Hypomethylated |
| | | | | high* | |
| BT-20 | Negative | Basal-like | Intermediate+ | Nd | hypomethylated |
| MDA-MB-231 | Negative | Basal-like | High | High* | hypomethylated |
| MDA-MET | Negative | Basal-like | High | High* | hypomethylated |

+Digital gene expression with primers used to perform qRT-PCR was applied to estimate gene expression.
Nd, Not determined.
*Flow cytometry data were published before (Cooney, Jousheghany et al. 2011).
++Flow cytometry was performed and no detectable binding of anti-CS-A mAb 2H6 was observed.

To confirm a role for methylation status of the CHST11 CpG island in the expression control of this gene, we further analyzed second generation DNA methylation sequencing data on breast cancer cell lines (Illumina Idea Challenge). This method provided information on DNA methylation at a single nucleotide resolution across 162 out of 186 CpGs in the ˜2.5 kb long CHST11 CpG island. Methylation analysis of Illumina Idea Challenge data of these cell lines indicate a hypermethylated CpG island of CHST11 gene in the T47-D and ZR-75-1 cell lines, similar to MCF7 cells (FIG. 11B,C,D). A very hypomethylated state was observed for the same sequence extracted from MDA-MB-231 cells (FIG. 11E). Therefore, the data attribute the negligible expression of CHST11 in these ER-positive cells to DNA hypermethylation.

Next, we examined the methylation status of another ER-positive cell line BT-474 and two triple-negative cell lines MDA-MB-468 and BT-20, both with epithelial appearance (FIG. 12). We also analyzed digital expression of mRNA-seq data on these cell lines and estimated the number of transcribed copies of CHST11 (C_{CHST11}), using the region covered by primers used for qRT-PCR (FIG. 12, LogEE, expression estimate, values). Consistent with what we observed with other ER-positive cells, the CpG island was highly methylated in BT-474 with a low level of expression of CHST11. Both BT-20 and MDA-MB-468 displayed a very hypomethylated state but the expression of CHST11 in both these cells was moderate and significantly less than what we observed for MDA-MB-231 and MDA-MET(Iida, Wilhelmson et al. 2007; Cooney, Jousheghany et al. 2011). BT-20 and MDA-MB-468 have epithelial appearance, while MDA-MB-231 and MDA-MET display mesenchymal appearance.

### CHST11 is Overexpressed in Basal-Like Cell Lines and ER-Negative Clinical Specimens.

Our data suggest that CHST11 expression is low in luminal cell lines and high in basal-like cell lines (Table 6). To further explore this possibility, we analyzed an Oncomine dataset that screened 50 breast cancer cell lines representative of molecular subtypes (Luminal, Her2-amplified, or Basal-like) (Hoeflich et al. 2009). We observed that the expression of CHST11 in basal-like cell lines is significantly higher than luminal cell types (FIG. 13A). CHST11 is also overexpressed in Her-2-amplified cell lines.

Our cell line-based data suggest that the expression of CHST11 is associated with ER status. We further analyzed the Gluck breast dataset (Gluck, Ross et al. 2011) and compared CHST11 expression levels between ER-negative and ER-positive specimens from breast cancer patients (FIG. 13B). The data are in harmony with the cell line expression analyses that suggest the expression of CHST11 is significantly higher in ER-negative specimens compared to ER-positive specimens. Further analysis of the data suggest that specimens with high expression of CHST11 are significantly more prevalent in ER-negative group (Fisher Exact Test, P=0.025).

### Discussion

We have previously shown that removal or blocking of CS on the surface of breast cancer cells inhibits metastasis (Monzavi-Karbassi, Stanley et al. 2007; Cooney, Jousheghany et al. 2011). We have demonstrated that the expression levels of CHST11 correlate with the expression of P-selectin-reactive surface CS and aggressiveness of human breast cancer cells (Cooney, Jousheghany et al. 2011). Here, we report that the CHST11 gene is overexpressed in cancer tissues and its overexpression correlates with progression and aggressive phenotypes of the disease. Therefore, studying the regulation CHST11 gene expression and its relationship to metastasis is relevant. Our data suggest that the expression can be controlled by DNA methylation. Aberrant DNA methylation is involved in tumor initiation and progression (Feinberg, Gehrke et al. 1988; Narayan, Ji et al. 1998; Baylin, Esteller et al. 2001; Ehrlich 2002; Szyf, Pakneshan et al. 2004; Cooney 2008). Hypermethylation of tumor suppressor genes is a common mechanism of gene silencing observed in cancer, and similarly, DNA hypomethylation can contribute to overexpression of tumor-promoting genes. DNA hypomethylation is associated with advanced stages, metastatic phenotypes, and drug-resistant variants of breast cancer (Soares et al. 1999; David et al. 2004; Pakneshan et al. 2004; Szyf, Pakneshan et al. 2004; Chekhun et al. 2006).

Methylation silencing of sulfotransferases, other than CHST11, is reported in breast cancer cell lines and clinical samples (Miyamoto, Asada et al. 2003). We find that CHST11 overexpression is accompanied by CHST11 gene hypomethylation and by increased levels of CHST11's immediate product, CS-A, in aggressive mesenchymal-like ER-negative breast cancer cell lines. Based upon our results, it seems that a combination of hypomethylation and high expression occurs in mesenchymal-like ER-negative cancer cells. In several ER-positive cell lines we found that CHST11 DNA is hypermethylated. Hypermethylation of CHST11 (and very low to no expression) clearly differentiates the least aggressive, ER-positive cells from the more aggressive ER-negative cells we tested. It needs to be pointed out that in less aggressive ER-negative cells that are epithelial-like (e.g. MDA-MB-468), hypomethylation is accompanied by intermediate expression of CHST11, suggesting involvement of other mechanisms in controlling the expression levels of this gene. It has been demonstrated that TGFβ1 can stimulate the expression of the CHST11 gene, implicating the TGF-β signaling pathway in expression control of this gene (Kluppel et al. 2002; Willis et al. 2009). However, using expression levels of CHST1 transcript, it might be possible to distinguish aggressive mesenchymal-like ER-negative cell lines, ie. MDA-MB-231, from less aggressive epithelial-like ER-negative cell lines, ie MDA-MB-468. Comparing the expression of epithelial and mesenchymal discriminators like vimentin, E-cadherin, and N-caderin, Blick and colleagues (Blick et al. 2008) found very similar pattern regarding vimentin expression levels in these two cell lines. Vimentin was overexpressed in MDA-MB-231 and its expression in MDA-MB-468 cells was moderate. The expression of E-cadherin or N-cadherin did not discriminate between these two cell lines. We examined several datasets in the Oncomine database for the expression of CHST11 and its relation to ER status or more aggressive molecular subtypes. We observed that overexpression is higher and more prevalent among ER-negative specimens and basal-like molecular subtype compared to ER-positive specimens and luminal cell lines, respectively. Others have shown that vimentin, the most widely used marker for mesenchymal phenotype, is absent in the majority of luminal cell lines but overexpressed in basal-like cell lines (Hugo et al. 2007; Blick, Widodo et al. 2008). Future studies on well-characterized clinical specimens are needed to determine the correlation between the expression of epithelial/mesenchymal markers and CHST11.

Our study illustrates an example of a gene, where methylation is lower and expression is higher as the cancer phenotype becomes more aggressive. In contrast, the main trend is widespread gene hypermethylation as cancers go from less aggressive to more aggressive phenotypes (Andrews et al. 2010; Wolff et al. 2010). Our data are consistent with some other models of metastatic and/or more aggressive cancers exemplifying tumor promoting genes like urokinase plasminogen activator (uPA), PAX3 and Ezrin that are less methylated and/or more highly expressed in the more aggressive cancer forms (Pakneshan, Szyf et al. 2004; Kurmasheva et al. 2005). This less frequent pattern of gene methylation between cancers with low and high aggressiveness suggests that the expression of these genes is necessary for the more aggressive phenotypes (because their change is counter to the trend).

The recognition that silencing of tumor suppressor genes through promoter hypermethylation plays a significant role in tumorigenesis (MacLeod et al. 1995; Ramchandani et al. 1997) has led to the clinical use of hypomethylating agents including 5-AzadC (Fenaux 2005). However, the expression of several pro-tumor genes is induced by DNA hypomethylation (Pakneshan, Szyf et al. 2004; Ehrlich 2006; Yu et al. 2010). The expression of such genes, including CHST11, may be activated by the clinical use of hypomethylating agents and this may promote more aggressive forms of breast cancer. In this regard, our data, in agreement with others (Szyf et al. 2004; Ateeq et al. 2008), suggest that therapeutic use of such demethylating agents may be effective in early developmental stages of breast cancer, but may promote tumor metastasis and recurrence later in the course of the disease. Histone deacetylase inhibitors can also hypomethylate genes and change their expression levels in breast cancer cell lines (Meeran et al. 2010) and their combination with metabolic therapies may modify their action (Cooney 2010). Therefore, additional studies are needed to determine if some agents or mechanisms of hypomethylation are more or less likely to promote tumor metastasis.

Epithelial-mesenchymal transition has been linked to cancer stem cells and ER-negative phenotypes (Mani et al. 2008; Blick et al. 2010; Jeong et al. 2012). An association between basal-like phenotype and cancer stem cells has been demonstrated (Honeth et al. 2008). Therefore, higher expression levels of CHST11 in mesenchymal versus epithelial cells, in basal-like versus luminal cells, or in ER-negative versus ER-positive cells and specimens may implicate CHST11 expression in the epithelial-mesenchymal transition and may also relate the expression of this gene to cancer stem cells (Thiery 2002; Al-Hajj et al. 2003; Mani, Guo et al. 2008). An increase in the expression of CHST11 may accompany cellular epithelial-mesenchymal transition programming and may serve as a prognostic/predictive biomarker or a therapeutic target for cancer stem cells. Interestingly the elevated expression is accompanied by the hypomethylated status of a CpG island, indicating that both methylation status and expression level, or a combination of two can be potentially employed to evaluate the patient outcome. We expect to validate these observations in clinical samples for correlation with hormone status, cancer stage and patient survival and response to therapy in future studies.

### Conclusions

We have shown that the expression of CHST11, which is a major determinant of CS-A, is modulated by DNA methylation. Therefore DNA methylation plays an important role in the remodeling of CS in breast tumors. Our data strongly suggest that the expression of CHST11 and its role in defining metastatic potential of tumor cells should be seriously considered when demethylating agents are used for medical treatment in breast cancer patients. Moreover, the methylation and expression of the CHST11 gene have potential as predictive and prognostic biomarkers. Given the importance of detection of circulating tumor cells in patients diagnosed with high stage breast cancer and predicting response in patients undergoing chemotherapy (Muller et al. 2006; Armstrong et al. 2011) a combination of methylation status and the expression of this gene could be valuable for the detection of circulating tumor cells and simultaneous characterization of patient response. Together, our data suggest that CHST11 can be targeted for development of novel prognostic/predictive biomarkers.

### Example 3

### CHST11 Overexpression in Other Cancers

We have analyzed CHST11 gene expression levels from matched tumor and nontumor tissue samples from several datasets to show that CHST11 expression level is significantly higher in several types of cancer than in the corresponding normal tissue.

FIG. 14 shows the CHST11 expression levels in 61 normal breast and 76 invasive breast adenocarcinoma samples. Expression levels are significantly higher in invasive breast carcinoma than normal breast. The dataset consists of Level 2 (processed) data from the TCGA data portal.

FIG. 15 shows CHST11 is overexpressed in cutaneous melanoma compared to normal skin. The dataset was 14 cutaneous melanoma samples and 4 normal skin.

We have also shown that CHST11 is overexpressed in cutaneous melanoma compared to melanocytic skin nevus (4.52 fold higher, P=3.49E-7). The data set was 45 cutaneous melanoma and 18 benign melanocytic skin nevus.

FIG. 16 shows CHST11 is overexpressed in oligodendroglyoma compared to normal brain. (Fold change of 1.99, P=5.74E-10). The data set was 23 normal brain samples and 50 oligodendroglioma samples.

The same CHST11 overexpression was observed in cercival squamous cell carcinoma compared to normal cervix. (Fold change 2.41, P=7.22E-5.) Forty cervical squamous cell carcinoma samples from 35 patients and 5 normal cervix samples were analyzed.

CHST11 overexpression was seen for esophagus cancer (Fold change 1.521, P=7.51E-12). Fifty-three esophageal squamous cell carcinoma samples and 53 adjacent paired normal esophagus samples were analyzed.

CHST11 was overexpressed in head and neck cancer (Fold change 4.073, P=5.03E-8). Forty-one head and neck squamous cell carcinoma and 13 buccal mucosa samples were analyzed on Affymetrix U133A microarrays.

The same CHST11 overexpression was observed for pancreatic cancer. (Fold change 3.061, P=6.02E-11.) Paired pancreatic ductal adenocarcinoma (n=39) and normal pancreas (n=39) samples from 36 patients were analyzed.

FIG. 17 shows CHST11 expression is associated with a basal-like molecular subtype in breast cancer.

FIG. 18 shows CHST11 expression is assacited with ER negative breast cancer.

We have also found that CHST11 overexpression correlates with occurrence of metastasis in breast cancer patients during 5-years post diagnosis. The CHST11 expression levels were compared between breast cancer patients that subsequently had no Metastatic Event at 5 Years (69 patients) and those with a Metastatic Event at 5 Years (6 patients). The data set was from Oncomime. Those that subsequently had a metastatic event had significantly higher CHST11 expression (Fold change 1.456, P=6.80E-4).

### REFERENCES

1. Hakomori S: Tumor malignancy defined by aberrant glycosylation and sphingo(glyco)lipid metabolism. Cancer Res 1996, 56:5309-5318.
2. Couldrey C, Green J E: Metastases: the glycan connection. Breast Cancer Res 2000, 2:321-323.
3. Gorelik E, Galili U, Raz A: On the role of cell surface carbohydrates and their binding proteins (lectins) in tumor metastasis. Cancer Metastasis Rev 2001, 20:245-277.
4. Kawaguchi T: Cancer metastasis: characterization and identification of the behavior of metastatic tumor cells and the cell adhesion molecules, including carbohydrates. Curr Drug Targets Cardiovasc Haematol Disord 2005, 5:39-64.
5. Korourian S, Siegel E, Kieber-Emmons T, Monzavi-Karbassi B: Expression analysis of carbohydrate antigens in ductal carcinoma in situ of the breast by lectin histochemistry. BMC Cancer 2008, 8:136.
6. Poole A R: Proteoglycans in health and disease: structures and functions. Biochem J 1986, 236:1-14.
7. Iozzo R V: Proteoglycans and neoplasia. Cancer Metastasis Rev 1988, 7:39-50.
8. Alini M, Losa G A: Partial characterization of proteoglycans isolated from neoplastic and normeoplastic human breast tissues. Cancer Res 1991, 51:1443-1447.
9. Vynios D H, Theocharis D A, Papageorgakopoulou N, Papadas T A, Mastronikolis N S, Goumas P D, Stylianou M, Skandalis S S: Biochemical changes of extracellular proteoglycans in squamous cell laryngeal carcinoma. Connect Tissue Res 2008, 49:239-243.
10. Stylianou M, Skandalis S S, Papadas T A, Mastronikolis N S, Theocharis D A, Papageorgakopoulou N, Vynios DH: Stage-related decorin and versican expression in human laryngeal cancer. Anticancer Res 2008, 28:245-251.
11. Kjellen L, Lindahl U: Proteoglycans: structures and interactions. Annu Rev Biochem 1991, 60:443-475.
12. Esko J D, Kimata K, Lindahl U: Proteoglycans and sulfated glycosaminoglycans. In Essentials of Glycobiology. Edited by Varki A, Cummings R D, Esko J D, Freeze H, Hart G, Marth J D. Cold Spring Harbor, N.Y.: Cold Spring Harbor Laboratory Press; 1999:653
13. Chiarugi V P, Dietrich C P: Sulfated mucopolysaccharides from normal and virus transformed rodent fibroblasts. J Cell Physiol 1979, 99:201-206.
14. Olsen E B, Trier K, Eldov K, Ammitzboll T: Glycosaminoglycans in human breast cancer. Acta Obstet Gynecol Scand 1988, 67:539-542.
15. Fthenou E, Zong F, Zafiropoulos A, Dobra K, Hjerpe A, Tzanakakis G N: Chondroitin sulfate A regulates fibrosarcoma cell adhesion, motility and migration through INK and tyrosine kinase signaling pathways. In Vivo 2009, 23:69-76.
16. Kim Y J, Borsig L, Varki N M, Varki A: P-selectin deficiency attenuates tumor growth and metastasis. Proc Natl Acad Sci USA 1998, 95:9325-9330.
17. Borsig L, Wong R, Feramisco J, Nadeau D R, Varki N M, Varki A: Heparin and cancer revisited: mechanistic connections involving platelets, P-selectin, carcinoma mucins, and tumor metastasis. Proc Natl Acad Sci USA 2001, 98:3352-3357.
18. Garcia J, Callewaert N, Borsig L: P-selectin mediates metastatic progression through binding to sulfatides on tumor cells. Glycobiology 2007, 17:185-196.
19. McEver R P: Selectin-carbohydrate interactions during inflammation and metastasis. Glycoconj J 1997, 14:585-591.
20. Aruffo A, Kolanus W, Walz G, Fredman P, Seed B: CD62/P-selectin recognition of myeloid and tumor cell sulfatides. Cell 1991, 67:35-44.
21. Needham L K, Schnaar R L: The HNK-1 reactive sulfoglucuronyl glycolipids are ligands for L-selectin and P-selectin but not E-selectin. Proc Natl Acad Sci USA 1993, 90:1359-1363.
22. Koenig A, Norgard-Sumnicht K, Linhardt R, Varki A: Differential interactions of heparin and heparan sulfate glycosaminoglycans with the selectins. Implications for the use of unfractionated and low molecular weight heparins as therapeutic agents. J Clin Invest 1998, 101:877-889.
23. Kawashima H, Atarashi K, Hirose M, Hirose J, Yamada S, Sugahara K, Miyasaka M: Oversulfated chondroitin/dermatan sulfates containing GlcAbeta1/IdoAalpha1-3GalNAc(4,6-O-disulfate) interact with L- and P-selectin and chemokines. J Biol Chem 2002, 277:12921-12930.
24. Monzavi-Karbassi B, Stanley J S, Hennings L, Jousheghany F, Artaud C, Shaaf S, Kieber-Emmons T: Chondroitin sulfate glycosaminoglycans as major P-selectin ligands on metastatic breast cancer cell lines. Int J Cancer 2007, 120:1179-1191.
25. Silbert J E, Sugumaran G: Biosynthesis of chondroitin/dermatan sulfate. IUBMB Life 2002, 54:177-186.
26. Sugahara K, Mikami T, Uyama T, Mizuguchi S, Nomura K, Kitagawa H: Recent advances in the structural biology of chondroitin sulfate and dermatan sulfate. Curr Opin Struct Biol 2003, 13:612-620.
27. Kusche-Gullberg M, Kjellen L: Sulfotransferases in glycosaminoglycan biosynthesis. Curr Opin Struct Biol 2003, 13:605-611.
28. Hiraoka N, Nakagawa H, Ong E, Akama T O, Fukuda M N, Fukuda M: Molecular cloning and expression of two distinct human chondroitin 4-O-sulfotransferases that belong to the HNK-1 sulfotransferase gene family. J Biol Chem 2000, 275:20188-20196.
29. Kang H G, Evers M R, Xia G, Baenziger J U, Schachner M: Molecular cloning and characterization of chondroitin-4-O-sulfotransferase-3. A novel member of the HNK-1 family of sulfotransferases. J Biol Chem 2002, 277:34766-34772.
30. Mikami T, Mizumoto S, Kago N, Kitagawa H, Sugahara K: Specificities of three distinct human chondroitin/dermatan N-acetylgalactosamine 4-O-sulfotransferases demonstrated using partially desulfated dermatan sulfate as an acceptor: implication of differential roles in dermatan sulfate biosynthesis. J Biol Chem 2003, 278:36115-36127.
31. Evers M R, Xia G, Kang H G, Schachner M, Baenziger J U: Molecular cloning and characterization of a dermatan-specific N-acetylgalactosamine 4-O-sulfotransferase. J Biol Chem 2001, 276:36344-36353.
32. Fukuta M, Kobayashi Y, Uchimura K, Kimata K, Habuchi O: Molecular cloning and expression of human chondroitin 6-sulfotransferase. Biochim Biophys Acta 1998, 1399:57-61.
33. Kitagawa H, Fujita M, Ito N, Sugahara K: Molecular cloning and expression of a novel chondroitin 6-O-sulfotransferase. J Biol Chem 2000, 275:21075-21080.
34. Habuchi O, Moroi R, Ohtake S: Enzymatic synthesis of chondroitin sulfate E by N-acetylgalactosamine 4-sulfate 6-O-sulfotransferase purified from squid cartilage. Anal Biochem 2002, 310:129-136.
35. Barbareschi M, Maisonneuve P, Aldovini D, Cangi M G, Pecciarini L, Angelo Mauri F, Veronese S, Caffo O, Lucenti A, Palma P D, Galligioni E, Doglioni C: High syndecan-1 expression in breast carcinoma is related to an aggressive phenotype and to poorer prognosis. Cancer 2003, 98:474-483.
36. Burbach B J, Friedl A, Mundhenke C, Rapraeger AC: Syndecan-1 accumulates in lysosomes of poorly differentiated breast carcinoma cells. Matrix Biol 2003, 22:163-177.
37. Baba F, Swartz K, van Buren R, Eickhoff J, Zhang Y, Wolberg W, Friedl A: Syndecan-1 and syndecan-4 are overexpressed in an estrogen receptor-negative, highly proliferative breast carcinoma subtype. Breast Cancer Res Treat 2006, 98:91-98.
38. Gotte M, Kersting C, Radke I, Kiesel L, Wulfing P: An expression signature of syndecan-1 (CD138), E-cadherin and c-met is associated with factors of angiogenesis and lymphangiogenesis in ductal breast carcinoma in situ. Breast Cancer Res 2007, 9:R8.
39. Wang X, Wang Y, Yu L, Sakakura K, Visus C, Schwab J H, Ferrone C R, Favoino E, Koya Y, Campoli M R, McCarthy J B, DeLeo A B, Ferrone S: CSPG4 in cancer: multiple roles. Curr Mol Med 2010, 10:419-429.
40. Bumol T F, Reisfeld R A: Unique glycoprotein-proteoglycan complex defined by monoclonal antibody on human melanoma cells. Proc Natl Acad Sci USA 1982, 79:1245-1249.
41. Nishiyama A, Dahlin K J, Prince J T, Johnstone S R, Stalicup W B: The primary structure of NG2, a novel membrane-spanning proteoglycan. J Cell Biol 1991, 114:359-371.
42. Stalicup W B: The NG2 antigen, a putative lineage marker: immunofluorescent localization in primary cultures of rat brain. Dev Biol 1981, 83:154-165.
43. Pluschke G, Vanek M, Evans A, Dittmar T, Schmid P, Itin P, Filardo E J, Reisfeld R A: Molecular cloning of a human melanoma-associated chondroitin sulfate proteoglycan. Proc Natl Acad Sci USA 1996, 93:9710-9715.
44. Burg M A, Grako K A, Stallcup W B: Expression of the NG2 proteoglycan enhances the growth and metastatic properties of melanoma cells. J Cell Physiol 1998, 177:299-312.
45. Yang J, Price M A, Li G Y, Bar-Eli M, Salgia R, Jagedeeswaran R, Carlson J H, Ferrone S, Turley E A, McCarthy J B: Melanoma proteoglycan modifies gene expression to stimulate tumor cell motility, growth, and epithelial-to-mesenchymal transition. Cancer Res 2009, 69:7538-7547.
46. Iida J, Wilhelmson K L, Ng J, Lee P, Morrison C, Tam E, Overall C M, McCarthy J B: Cell surface chondroitin sulfate glycosaminoglycan in melanoma: role in the activation of pro-MMP-2 (pro-gelatinase A). Biochem J2007, 403:553-563.
47. Wilson B S, Imai K, Natali P G, Ferrone S: Distribution and molecular characterization of a cell-surface and a cytoplasmic antigen detectable in human melanoma cells with monoclonal antibodies. Int J Cancer 1981, 28:293-300.
48. Temponi M, Gold A M, Ferrone S: Binding parameters and idiotypic profile of the whole immunoglobulin and Fab′ fragments of murine monoclonal antibody to distinct determinants of the human high molecular weight-melanoma associated antigen. Cancer Res 1992, 52:2497-2503.
49. Bendre M S, Gaddy-Kurten D, Mon-Foote T, Akel N S, Skinner R A, Nicholas R W, Suva L J: Expression of interleukin 8 and not parathyroid hormone-related protein by human breast cancer cells correlates with bone metastasis in vivo. Cancer Res 2002, 62:5571-5579.
50. Luo W, Hsu J C, Tsao C Y, Ko E, Wang X, Ferrone S: Differential immunogenicity of two peptides isolated by high molecular weight-melanoma-associated antigen-specific monoclonal antibodies with different affinities. J Immunol 2005, 174:7104-7110.
51. Livak K J, Schmittgen T D: Analysis of relative gene expression data using real-time quantitative PCR and the 2(−Delta Delta C(T)) Method. Methods 2001, 25:402-408.
52. Monzavi-Karbassi B, Whitehead T L, Jousheghany F, Artaud C, Hennings L, Shaaf S, Slaughter A, Korourian S, Kelly T, Blaszczyk-Thurin M, Kieber-Emmons T: Deficiency in surface expression of E-selectin ligand promotes lung colonization in a mouse model of breast cancer. Int J Cancer 2005, 117:398-408.
53. Faassen A E, Schrager J A, Klein D J, Oegema T R, Couchman J R, McCarthy J B: A cell surface chondroitin sulfate proteoglycan, immunologically related to CD44, is involved in type I collagen-mediated melanoma cell motility and invasion. J Cell Biol 1992, 116:521-531.
54. Deepa S S, Yamada S, Zako M, Goldberger O, Sugahara K: Chondroitin sulfate chains on syndecan-1 and syndecan-4 from normal murine mammary gland epithelial cells are structurally and functionally distinct and cooperate with heparan sulfate chains to bind growth factors. A novel function to control binding of midkine, pleiotrophin, and basic fibroblast growth factor. J Biol Chem 2004, 279:37368-37376.
55. Shintani Y, Takashima S, Asano Y, Kato H, Liao Y, Yamazaki S, Tsukamoto O, Seguchi O, Yamamoto H, Fukushima T, Sugahara K, Kitakaze M, Hori M: Glycosaminoglycan modification of neuropilin-1 modulates VEGFR2 signaling. Embo J 2006, 25:3045-3055.
56. Bielenberg D R, Pettaway C A, Takashima S, Klagsbrun M: Neuropilins in neoplasms: expression, regulation, and function. Exp Cell Res 2006, 312:584-593.
57. Uyama T, Ishida M, Izumikawa T, Trybala E, Tufaro F, Bergstrom T, Sugahara K, Kitagawa H: Chondroitin 4-O-sulfotransferase-1 regulates E disaccharide expression of chondroitin sulfate required for herpes simplex virus infectivity. J Biol Chem 2006, 281:38668-38674.
58. Li F, ten Dam G B, Murugan S, Yamada S, Hashiguchi T, Mizumoto S, Oguri K, Okayama M, van Kuppevelt T H, Sugahara K: Involvement of highly sulfated chondroitin sulfate in the metastasis of the Lewis lung carcinoma cells. J Biol Chem 2008, 283:34294-34304.
59. Basappa, Murugan S, Sugahara K N, Lee C M, ten Dam G B, van Kuppevelt T H, Miyasaka M, Yamada S, Sugahara K: Involvement of chondroitin sulfate E in the liver tumor focal formation of murine osteosarcoma cells. Glycobiology 2009, 19:735-742.
60. Potapenko I O, Haakensen V D, Luders T, Helland A, Bukholm I, Sorlie T, Kristensen V N, Lingjaerde O C, Borresen-Dale A L: Glycan gene expression signatures in normal and malignant breast tissue; possible role in diagnosis and progression. Mol Oncol 2010, 4:98-118.
61. Bret C, Hose D, Reme T, Sprynski A C, Mahtouk K, Schved J F, Quittet P, Rossi J F, Goldschmidt H, Klein B: Expression of genes encoding for proteins involved in heparan sulphate and chondroitin sulphate chain synthesis and modification in normal and malignant plasma cells. Br J Haematol 2009, 145:350-368.
62. Campoli M R, Chang C C, Kageshita T, Wang X, McCarthy J B, Ferrone S: Human high molecular weight-melanoma-associated antigen (HMW-MAA): a melanoma cell surface chondroitin sulfate proteoglycan (MSCP) with biological and clinical significance. Crit. Rev Immunol 2004, 24:267-296.
Al-Hajj, M., Wicha, M. S., Benito-Hernandez, A., Morrison, S. J. and Clarke, M. F. (2003). Prospective identification of tumorigenic breast cancer cells. Proc Natl Acad Sci USA 100, 3983-3988.
Andrews, J., Kennette, W., Pilon, J., Hodgson, A., Tuck, A. B., Chambers, A. F. and Rodenhiser, D. I. (2010). Multi-platform whole-genome microarray analyses refine the epigenetic signature of breast cancer metastasis with gene expression and copy number. PLoS One 5, e8665.
Armstrong, A. J., Marengo, M. S., Oltean, S., Kemeny, G., Bitting, R. L., Turnbull, J. D., Herold, C. I., Marcom, P. K., George, D. J. and Garcia-Blanco, M. A. (2011). Circulating tumor cells from patients with advanced prostate and breast cancer display both epithelial and mesenchymal markers. Mol Cancer Res 9, 997-1007.
Ateeq, B., Unterberger, A., Szyf, M. and Rabbani, S. A. (2008). Pharmacological inhibition of DNA methylation induces proinvasive and prometastatic genes in vitro and in vivo. Neoplasia 10, 266-278.
Baylin, S. B., Esteller, M., Rountree, M. R., Bachman, K. E., Schuebel, K. and Herman, J. G. (2001). Aberrant patterns of DNA methylation, chromatin formation and gene expression in cancer. Hum Mol Genet. 10, 687-692.
Buick, T., Hugo, H., Widodo, E., Waltham, M., Pinto, C., Mani, S. A., Weinberg, R. A., Neve, R. M., Lenburg, M. E. and Thompson, E. W. (2010). Epithelial mesenchymal transition traits in human breast cancer cell lines parallel the CD44(hi/)CD24 (lo/−) stem cell phenotype in human breast cancer. J Mammary Gland Biol Neoplasia 15, 235-252.
Blick, T., Widodo, E., Hugo, H., Waltham, M., Lenburg, M. E., Neve, R. M. and Thompson, E. W. (2008). Epithelial mesenchymal transition traits in human breast cancer cell lines. Clin Exp Metastasis 25, 629-642.
Bui, C., Ouzzine, M., Talhaoui, I., Sharp, S., Prydz, K., Coughtrie, M. W. and Fournel-Gigleux, S. (2010). Epigenetics: methylation-associated repression of heparan sulfate 3-O-sulfotransferase gene expression contributes to the invasive phenotype of H-EMC-SS chondrosarcoma cells. Faseb J 24, 436-450.
Chekhun, V. F., Kulik, G. I., Yurchenko, O. V., Tryndyak, V. P., Todor, I. N., Luniv, L. S., Tregubova, N. A., Pryzimirska, T. V., Montgomery, B., Rusetskaya, N. V. and Pogribny, I. P. (2006). Role of DNA hypomethylation in the development of the resistance to doxorubicin in human MCF-7 breast adenocarcinoma cells. Cancer Lett 231, 87-93.
Cooney, C. A. (2008). Cancer and Aging: The Epigenetic Connection. Cancer Epigenetics. T. Tollefsbol. Boca Raton, Fla., CRC Press: 303-316.
Cooney, C. A. (2010). Drugs and Supplements that May Slow Aging of the Epigenome. Drug Discovery Today: Therapeutic Strategies 7, 57-84.
Cooney, C. A., Jousheghany, F., Yao-Borengasser, A., Phanavanh, B., Gomes, T., Kieber-Emmons, A. M., Siegel, E. R., Suva, L. J., Ferrone, S., Kieber-Emmons, T. and Monzavi-Karbassi, B. (2011). Chondroitin sulfates play a major role in breast cancer metastasis: a role for CSPG4 and CHST11 gene expression in forming surface P-selectin ligands in aggressive breast cancer cells. Breast Cancer Res 13, R58.
David, G. L., Yegnasubramanian, S., Kumar, A., Marchi, V. L., De Marzo, A. M., Lin, X. and Nelson, W. G. (2004). MDR1 promoter hypermethylation in MCF-7 human breast cancer cells: changes in chromatin structure induced by treatment with 5-Aza-cytidine. Cancer Biol Ther 3, 540-548.
Ehrlich, M. (2002). DNA methylation in cancer: too much, but also too little. Oncogene 21, 5400-5413.
Ehrlich, M. (2006). Cancer-linked DNA hypomethylation and its relationship to hypermethylation. Curr Top Microbiol Immunol 310, 251-274.
Feinberg, A. P., Gehrke, C. W., Kuo, K. C. and Ehrlich, M. (1988). Reduced genomic 5-methylcytosine content in human colonic neoplasia. Cancer Res 48, 1159-1161.
Fenaux, P. (2005). Inhibitors of DNA methylation: beyond myelodysplastic syndromes. Nat Clin Pract Oncol 2 Suppl 1, S36-44.
Gluck, S., Ross, J. S., Royce, M., McKenna, E. F., Jr., Perou, C. M., Avisar, E. and Wu, L. (2011). TP53 genomics predict higher clinical and pathologic tumor response in operable early-stage breast cancer treated with docetaxel-capecitabine+/−trastuzumab. Breast Cancer Res Treat.
Hoeflich, K. P., O'Brien, C., Boyd, Z., Cavet, G., Guerrero, S., Jung, K., Januario, T., Savage, H., Punnoose, E., Truong, T., Zhou, W., Berry, L., Murray, L., Amler, L., Belvin, M., Friedman, L. S, and Lackner, M. R. (2009). In vivo antitumor activity of MEK and phosphatidylinositol 3-kinase inhibitors in basal-like breast cancer models. Clin Cancer Res 15, 4649-4664.
Honeth, G., Bendahl, P. O., Ringner, M., Saal, L. H., Gruvberger-Saal, S. K., Lovgren, K., Grabau, D., Ferno, M., Borg, A. and Hegardt, C. (2008). The CD44+/CD24− phenotype is enriched in basal-like breast tumors. Breast Cancer Res 10, R53.
Hugo, H., Ackland, M. L., Blick, T., Lawrence, M. G., Clements, J. A., Williams, E. D. and Thompson, E. W. (2007). Epithelial—mesenchymal and mesenchymal—epithelial transitions in carcinoma progression. J Cell Physiol 213, 374-383.
Iida, J., Wilhelmson, K. L., Ng, J., Lee, P., Morrison, C., Tam, E., Overall, C. M. and McCarthy, J. B. (2007). Cell surface chondroitin sulfate glycosaminoglycan in melanoma: role in the activation of pro-MMP-2 (pro-gelatinase A). Biochem J 403, 553-563.
Jeong, H., Ryu, Y. J., An, J., Lee, Y. and Kim, A. (2012). Epithelial-mesenchymal transition in breast cancer correlates with high histological grade and triple-negative phenotype. Histopathology.
Kalathas, D., Triantaphyllidou, I. E., Mastronikolis, N. S., Goumas, P. D., Papadas, T. A., Tsiropoulos, G. and Vynios, D. H. (2010). The chondroitin/dermatan sulfate synthesizing and modifying enzymes in laryngeal cancer: expressional and epigenetic studies. Head Neck Oncol 2, 27.
Kluppel, M., Vallis, K. A. and Wrana, J. L. (2002). A high-throughput induction gene trap approach defines C4ST as a target of BMP signaling. Mech Dev 118, 77-89.
Kurmasheva, R. T., Peterson, C. A., Parham, D. M., Chen, B., McDonald, R. E. and Cooney, C. A. (2005). Upstream CpG island methylation of the PAX3 gene in human rhabdomyosarcomas. Pediatr Blood Cancer 44, 328-337.
Leakey, T. I., Zielinski, J., Siegfried, R. N., Siegel, E. R., Fan, C. Y. and Cooney, C. A. (2008). A simple algorithm for quantifying DNA methylation levels on multiple independent CpG sites in bisulfite genomic sequencing electropherograms. Nucleic Acids Res 36, e64.
Li, F., Ten Dam, G. B., Murugan, S., Yamada, S., Hashiguchi, T., Mizumoto, S., Oguri, K., Okayama, M., van Kuppevelt, T. H. and Sugahara, K. (2008). Involvement of highly sulfated chondroitin sulfate in the metastasis of the Lewis lung carcinoma cells. J Biol Chem 283, 34294-34304.
Li, L. C. and Dahiya, R. (2002). MethPrimer: designing primers for methylation PCRs. Bioinformatics 18, 1427-1431.
Livak, K. J. and Schmittgen, T. D. (2001). Analysis of relative gene expression data using real-time quantitative PCR and the 2(−Delta Delta C(T)) Method. Methods 25, 402-408.
MacLeod, A. R., Rouleau, J. and Szyf, M. (1995). Regulation of DNA methylation by the Ras signaling pathway. J Biol Chem 270, 11327-11337.
Mani, S. A., Guo, W., Liao, M. J., Eaton, E. N., Ayyanan, A., Zhou, A. Y., Brooks, M., Reinhard, F., Zhang, C. C., Shipitsin, M., Campbell, L. L., Polyak, K., Brisken, C., Yang, J. and Weinberg, R. A. (2008). The epithelial-mesenchymal transition generates cells with properties of stem cells. Cell 133, 704-715.
Meeran, S. M., Patel, S, N. and Tollefsbol, T. O. (2010). Sulforaphane causes epigenetic repression of hTERT expression in human breast cancer cell lines. PLoS One 5, e11457.
Meissner, A., Mikkelsen, T. S., Gu, H., Wernig, M., Hanna, J., Sivachenko, A., Zhang, X., Bernstein, B. E., Nusbaum, C., Jaffe, D. B., Gnirke, A., Jaenisch, R. and Lander, E. S. (2008). Genome-scale DNA methylation maps of pluripotent and differentiated cells. Nature 454, 766-770.
Mikami, T., Mizumoto, S., Kago, N., Kitagawa, H. and Sugahara, K. (2003). Specificities of three distinct human chondroitin/dermatan N-acetylgalactosamine 4-O-sulfotransferases demonstrated using partially desulfated dermatan sulfate as an acceptor: implication of differential roles in dermatan sulfate biosynthesis. J Biol Chem 278, 36115-36127.
Miyamoto, K., Asada, K., Fukutomi, T., Okochi, E., Yagi, Y., Hasegawa, T., Asahara, T., Sugimura, T. and Ushijima, T. (2003). Methylation-associated silencing of heparan sulfate D-glucosaminyl 3-O-sulfotransferase-2 (3-OST-2) in human breast, colon, lung and pancreatic cancers. Oncogene 22, 274-280.
Monzavi-Karbassi, B., Stanley, J. S., Hennings, L., Jousheghany, F., Artaud, C., Shaaf, S. and

Kieber-Emmons, T. (2007). Chondroitin sulfate glycosaminoglycans as major P-selectin ligands on metastatic breast cancer cell lines. Int J Cancer 120, 1179-1191.
Mortazavi, A., Williams, B. A., McCue, K., Schaeffer, L. and Wold, B. (2008). Mapping and quantifying mammalian transcriptomes by RNA-Seq. Nat. Methods 5, 621-628.
Muller, A., Sonkoly, E., Eulert, C., Gerber, P. A., Kubitza, R., Schirlau, K., Franken-Kunkel, P., Poremba, C., Snyderman, C., Klotz, L. O., Ruzicka, T., Bier, H., Zlotnik, A., Whiteside, T. L., Homey, B. and Hoffmann, T. K. (2006). Chemokine receptors in head and neck cancer: association with metastatic spread and regulation during chemotherapy. Int J Cancer 118, 2147-2157.
Narayan, A., Ji, W., Zhang, X. Y., Marrogi, A., Graff, J. R., Baylin, S. B. and Ehrlich, M. (1998). Hypomethylation of pericentromeric DNA in breast adenocarcinomas. Int J Cancer 77, 833-838.
Pakneshan, P., Szyf, M., Farias-Eisner, R. and Rabbani, S. A. (2004). Reversal of the hypomethylation status of urokinase (uPA) promoter blocks breast cancer growth and metastasis. J Biol Chem 279, 31735-31744.
Ramchandani, S., MacLeod, A. R., Pinard, M., von Hofe, E. and Szyf, M. (1997). Inhibition of tumorigenesis by a cytosine-DNA, methyltransferase, antisense oligodeoxynucleotide. Proc Natl Acad Sci USA 94, 684-689.
Rhodes, D. R., Yu, J., Shanker, K., Deshpande, N., Varambally, R., Ghosh, D., Barrette, T., Pandey, A. and Chinnaiyan, A. M. (2004). ONCOMINE: a cancer microarray database and integrated data-mining platform. Neoplasia 6, 1-6.
Schuetz, C. S., Bonin, M., Clare, S. E., Nieselt, K., Sotlar, K., Walter, M., Fehm, T., Solomayer, E., Riess, O., Wallwiener, D., Kurek, R. and Neubauer, H. J. (2006). Progression-specific genes identified by expression profiling of matched ductal carcinomas in situ and invasive breast tumors, combining laser capture microdissection and oligonucleotide microarray analysis. Cancer Res 66, 5278-5286.
Smith, Z. D., Gu, H., Bock, C., Gnirke, A. and Meissner, A. (2009). High-throughput bisulfite sequencing in mammalian genomes. Methods 48, 226-232.
Soares, J., Pinto, A. E., Cunha, C. V., Andre, S., Barao, I., Sousa, J. M. and Cravo, M. (1999). Global DNA hypomethylation in breast carcinoma: correlation with prognostic factors and tumor progression. Cancer 85, 112-118.
Szyf, M., Pakneshan, P. and Rabbani, S. A. (2004). DNA demethylation and cancer: therapeutic implications. Cancer Lett 211, 133-143.
Szyf, M., Pakneshan, P. and Rabbani, S. A. (2004). DNA methylation and breast cancer. Biochem Pharmacol 68, 1187-1197.
Takai, D. and Jones, P. A. (2003). The CpG island searcher: a new WWW resource. In Silico Biol 3, 235-240.
Thiery, J. P. (2002). Epithelial-mesenchymal transitions in tumour progression. Nat Rev Cancer 2, 442-454.
Tokuyama, Y., Takahashi, T., Okumura, N., Nonaka, K., Kawaguchi, Y., Yamaguchi, K., Osada, S., Gazdar, A. and Yoshida, K. (2010). Aberrant methylation of heparan sulfate glucosamine 3-O-sulfotransferase 2 genes as a biomarker in colorectal cancer. Anticancer Res 30, 4811-4818.
Uyama, T., Ishida, M., Izumikawa, T., Trybala, E., Tufaro, F., Bergstrom, T., Sugahara, K. and Kitagawa, H. (2006). Chondroitin 4-O-sulfotransferase-1 regulates E disaccharide expression of chondroitin sulfate required for herpes simplex virus infectivity. J Biol Chem 281, 38668-38674.
Willis, C. M., Wrana, J. L. and Kluppel, M. (2009). Identification and characterization of TGFbeta-dependent and -independent cis-regulatory modules in the C4ST-1/CHST11 locus. Genet Mol Res 8, 1331-1343.
Wolff, E. M., Chihara, Y., Pan, F., Weisenberger, D. J., Siegmund, K. D., Sugano, K., Kawashima, K., Laird, P. W., Jones, P. A. and Liang, G. (2010). Unique DNA methylation patterns distinguish noninvasive and invasive urothelial cancers and establish an epigenetic field defect in premalignant tissue. Cancer Res 70, 8169-8178.
Yu, Y., Zeng, P., Xiong, J., Liu, Z., Berger, S. L. and Merlino, G. (2010). Epigenetic drugs can stimulate metastasis through enhanced expression of the pro-metastatic Ezrin gene. PLoS One 5, e12710.

All patents, patent publications, and other references cited are hereby incorporated by reference.

What is claimed is:

1. A method of determining a prognosis of a cancer in a human comprising:
determining expression level of CHST11 in a cancer tissue sample or determining methylation status of CHST11 gene in a cancer tissue sample.

2. The method of claim 1 wherein the method comprises determining methylation status of CHST11 gene in a tissue sample.

3. The method of claim 1 wherein the tissue sample is a blood sample, an enriched circulating tumor cell sample, or a tumor biopsy sample.

4. The method of claim 1 wherein the sample is a tumor biopsy sample.

5. The method of claim 4 wherein the sample is a living or frozen tumor biopsy sample.

6. The method of claim 4 wherein the sample is a paraffin-embedded tumor biopsy sample.

7. The method of claim 1 wherein the cancer is breast cancer, an epithelial cancer, osteosarcoma, brain cancer, or a blood cancer.

8. The method of claim 1 wherein the cancer is melanoma, cervical cancer, esophagus cancer, head and neck cancer, or pancreatic cancer.

9. The method of claim 1 further comprising determining expression level of CSPG4 in the cancer tissue sample or determining methylation status of CSPG4 gene in a cancer tissue sample.

10. The method of claim 9 wherein the method comprises determining methylation status of CSPG4 gene in a cancer tissue sample.

11. The method of claim 2 wherein the method further comprises determining methylation status of CSPG4 gene in a cancer tissue sample.

12. The method of claim 11 wherein the tissue sample is paraffin-embedded tumor biopsy sample.

13. The method of claim 11 wherein the tissue sample is a living or frozen tumor biopsy sample.


SEQUENCE LISTING (39 sequences)

SEQ ID NO: 1 - DNA, 20 bp, homo sapiens
ttcgaacgtctgccctatca

SEQ ID NO: 2 - DNA, 19 bp, Homo sapiens
atggtaggcacggcgacta

SEQ ID NO: 3 - DNA, 23 bp, Homo sapiens
gacttcgagcaagagatggccac

SEQ ID NO: 4 - DNA, 23 bp, Homo sapiens
caatgccagggtacatggtggtg

SEQ ID NO: 5 - DNA, 20 bp, Homo sapiens
gactttgtgcacagcctgaa

SEQ ID NO: 6 - DNA, 20 bp, Homo sapiens
agagcttggggaatctgctt

SEQ ID NO: 7 - DNA, 20 bp, Homo sapiens
ggggcaatctgtcacactct

SEQ ID NO: 8 - DNA, 20 bp, Homo sapiens
aattgcacagcagttgttgg

SEQ ID NO: 9 - DNA, 20 bp, Homo sapiens
tccctttggtgtggacatct

SEQ ID NO: 10 - DNA, 20 bp, Homo sapiens
cacgtgtctgtcacctggtc

SEQ ID NO: 11 - DNA, 20 bp, Homo sapiens
gcacacgtccttctctaggc

SEQ ID NO: 12 - DNA, 20 bp, Homo sapiens
aaactcgtcgacatcggagt

SEQ ID NO: 13 - DNA, 19 bp, Homo sapiens
ccggcatttggaaacagag

SEQ ID NO: 14 - DNA, 20 bp, Homo sapiens
gggtcctgatccaggtcata

SEQ ID NO: 15 - DNA, 20 bp, Homo sapiens
tctgatcccccatttatcca

SEQ ID NO: 16 - DNA, 20 bp, Homo sapiens
ggagcagaaaggcacaaaag

SEQ ID NO: 17 - DNA, 20 bp, Homo sapiens
tgagttcacgaccagacagc

SEQ ID NO: 18 - DNA, 20 bp, Homo sapiens
cgtaccaacaggggctctta

SEQ ID NO: 19 - DNA, 20 bp, Homo sapiens
cacacagaggaaccctcgat

SEQ ID NO: 20 - DNA, 20 bp, Homo sapiens
cttcagcgagaggagcactt

SEQ ID NO: 21 - DNA, 21 bp, Homo sapiens
aggtgggatacgaaaagaccg

SEQ ID NO: 22 - DNA, 21 bp, Homo sapiens
aaggttggcagctctcatgtc

SEQ ID NO: 23 - DNA, 20 bp, Homo sapiens
acagtcagccgcatcttctt

SEQ ID NO: 24 - DNA, 20 bp, Homo sapiens
acgaccaaatccgttgactc

SEQ ID NO: 25 - DNA, 24 bp, Homo sapiens
caaggtgttcatgaggaagttcaa

SEQ ID NO: 26 - DNA, 23 bp, Homo sapiens
ccgcagctcaggtgtatcatagt

SEQ ID NO: 27 - DNA, 20 bp, Homo sapiens
gctctggggatgactctgac

SEQ ID NO: 28 - DNA, 20 bp, Homo sapiens
ggtctgctgtgacaaggtga

SEQ ID NO: 29 - DNA, 20 bp, Homo sapiens
gtctggctctggagatctgg

SEQ ID NO: 30 - DNA, 20 bp, Homo sapiens
caccaagggatggacaactt

SEQ ID NO: 31 - DNA, 21 bp, Homo sapiens
ucaguaguucucguagacutt

SEQ ID NO: 32 - DNA, 21 bp, Homo sapiens
uucugggugaacuuguugcgg

SEQ ID NO: 33 - DNA, 21 bp, Homo sapiens
aucgaguuuguagacuucgta

SEQ ID NO: 34 - DNA, 27 bp, Homo sapiens
tttgattattgtagttttggaggaaat

SEQ ID NO: 35 - DNA, 30 bp, Homo sapiens
ccttacaattaaaaaaacaaattattacta

SEQ ID NO: 36 - DNA, 25 bp, Homo sapiens
tttttgtggtttaggtaaagtttta

SEQ ID NO: 37 - DNA, 351 bp, Homo sapiens
cccttgtggctcaggcaaagttccacgtccgaaatctggactgggggagggacgaggctcgtcgcttcctaggggtgcgagggaaagtttgggttctggagagggaggggcgccctcgagcgcggcggccgggggtgagcagctcggctgcgaagcccaacaggtagaacgttccgagaagccttccgggtaacgcgggtctccccgccaagccgtggctcccctgctcccttttaccctcccttagcagccccctgccgggccacgttggctcagaaatccattttctacgcctccgagttgcctttctcagcgtagtcacagcaataatttgcttttctaattgcaagg

SEQ ID NO: 38 - DNA, 44 bp, Homo sapiens
tttttcgggtaacgcgggttttttcgttaagtcgtggttttttt

SEQ ID NO: 39 - DNA, 44 bp, Homo sapiens
ttttttgggtaatgtgggtttttttgttaagttgtggttttttt